(19) United States
(12) Patent Application Publication
(10) Pub. No.: US 2003/0187059 A1
(43) Pub. Date: Oct. 2, 2003

(54) Methods and compounds useful in inhibiting oxidative and/or free radical damage and in the treatment and prevention of disease

(76) Inventors: Robert M. Levin, Albany, NY (US); Martha A. Hass, Wynantskill, NY (US)

Correspondence Address: Peter Rogalskyj, Esq., Braman & Rogalskyj, LLP, P.O. Box 352, Canandaigua, NY, 14424-0352, US

(21) Appl. No.: 10/373,625

(22) Filed: Feb. 24, 2003

Related U.S. Application Data
(60) Provisional application No. 60/359,080, filed on Feb. 22, 2002.
(60) Provisional application No. 60/387,943, filed on Jun. 12, 2002.

(51) Int. Cl. A61K 31/382; C 07D 4 9/02; A61K 31/355; A61K 31/385

(52) U.S. Cl. 514/440; 514/458; 549/35; 549/408

(57) ABSTRACT

Disclosed are methods for treating obstructive and ischemic bladder diseases which include administering a compound which includes a cyclic or acyclic disulfide covalently bonded to a lipid-soluble antioxidant or which include administering a reduced sulfhydryl derivative thereof. Also disclosed are compounds that include a benzopyran moiety which is directly or indirectly covalently bonded to a cyclic or acyclic disulfide, as well as reduced sulfhydryl derivatives of such compounds. Methods for making such compounds and reduced sulfhydryl derivatives using tocopherol and lipoic acid starting materials are also disclosed, as are methods for inhibiting oxidative and/or free radical damage in a subject's cells, nerve membranes, sarcoplasmic reticula, mitochondrial membranes, and/or muscle plasma membranes and methods for treating and/or preventing obstructive and ischemic bladder diseases, conditions involving hypoxia, ischemia, and/or reoxygenation injury.

The present application claims the benefit of U.S. Provisional Patent Application Serial No. 60/359,080, filed Feb. 22, 2002, and U.S. Provisional Patent Application Serial No. 60/387,943, filed Jun. 12, 2002, each of which provisional patent applications is hereby incorporated by reference.

### FIELD OF THE INVENTION

The present invention relates, in part, to methods and compounds that are useful in inhibiting oxidative and/or free radical damage and in the treatment and prevention of disease, particularly, obstructive and ischemic bladder diseases and other diseases involving ischemia, hypoxia, and reoxygenation injury.

### BACKGROUND OF THE INVENTION

Bladder dysfunction secondary to benign prostatic hyperplasia (“BPH”) is a major affliction associated with human aging (Girman et al., “Epidemiology of Benign Prostatic Hyperplasia,” pp. 116-126 in Lepor, ed., Prostatic Disease, Philadelphia, Pa.: W. B. Saunders Co. (2000); Barry et al., “The Natural History of Benign Prostatic Hyperplasia,” pp. 106-115 in Lepor, ed., Prostatic Disease, Philadelphia, Pa.: W. B. Saunders Co. (2000); and Boyle et al., “Epidemiology and Natural History,” pp 19-68 in Chatelain et al., Benign Prostatic Hyperplasia (5th International Consultation on Benign Prostatic Hyperplasia), Plymouth, U.K.: Plymbridge Distributors, Ltd. (2001)). It is clear that bladder dysfunction secondary to BPH is a slow progressive disease. In many cases, medical treatment is not sought until the dysfunction is relatively severe. This is primarily a function of the insidious nature of the disease. It is well known that bladder function can remain relatively “normal” for many years during the progression of BPH. This is because the bladder can compensate for the progressive increase in urethral resistance (mediated by prostate growth) by bladder hypertrophy (an increase in bladder wall thickness and mass). During this compensated period of functioning, there are changes in micturition pressure and flow characteristics, these changes are not severe and, therefore, do not require medical attention. It is not until the patient shifts to decompensated function that severe alterations occur, and the patient seeks medical attention. These clinical changes leave the patient susceptible to subsequent renal injury and frequent urinary infections in addition to the considerable discomfort experienced prior to and during urination.

In man, it is difficult to investigate the cellular mechanisms by which progressive bladder dysfunction occurs. However, many of the functional changes associated with human bladder pathology can be induced in experimental animal model systems. This has been demonstrated prominently in a rabbit model of partial bladder outlet obstruction, where a partial outlet obstruction is created surgically by placing a ligature loosely around the urethra. See, for example, the reviews set forth in Levin et al., “Rabbit as a Model of Urinary Bladder Function,” Neurourol. Urodyn., 13:119-135 (1994); Levin et al., “Genetic and Cellular Characteristics of Bladder Outlet Obstruction,” Urol. Clin. North Am., 22:263-283 (1995); Levin et al., “Experimental Models of Bladder Outlet Obstruction,” pp. 119-130 in Lepor et al., eds., Prostate Diseases, Philadelphia, Pa.: W. B. Saunders Co. (1993); and Levin et al., “Cellular and Molecular Aspects of Bladder Hypertrophy,” Eur. Urol., 32(supp):15-21 (1997).

The progressive response to partial outlet obstruction can be divided into three distinct phases. The first phase involves an initial response to surgical induction of partial outlet obstruction (days 1-14) characterized by bladder dilation followed by a progressive increase in bladder mass and specific phasic contractile and metabolic dysfunctions. The second phase involves compensated bladder function and immediately follows the “initial phase”. The second phase lasts an indefinite and variable length of time, and it is characterized by relatively stable bladder mass and function and by relatively stable contractile responses to field stimulation (“FS”), bethanechol stimulation, and KCl stimulation. However, during this second phase, there are progressive morphological changes in bladder cell structure. At some point, the functional ability to contract and empty degenerates, and the bladder becomes “decompensated”. This marks the onset of the third phase, which is also referred to as the decompensated phase. This phase is characterized by progressive deterioration in contractility and function (i.e., ability to generate pressure and empty), a further increase in mass, and a progressive decrease in the volume fraction of smooth muscle elements within the bladder wall. The end result is either an organ with a thick fibrous wall, low capacity, poor compliance, and little or no contractile function; or a dilated bladder with a thin fibrous wall, high capacity, and little or no contractile function. Further details regarding the three phases of partial outlet obstruction can be found, for example, in Kato et al., “The Functional Effects of Longterm Outlet Obstruction on the Rabbit Urinary Bladder,” J. Urol., 143:600-606 (1990) (“Kato”) and in Levin et al., “Studies on Experimental Bladder Outlet Obstruction in the Cat: Long-term Functional Effects,” J. Urol., 148:939-943 (1992) (“Levin I”).

Although much has been done to understand the functional changes associated with human bladder pathology, a need continues to exist for methods for preventing and treating bladder dysfunction secondary to BPH. The present invention is directed, in part, to meeting this need.

### SUMMARY OF THE INVENTION

The present invention relates to compounds which include a cyclic or acyclic disulfide that is covalently bonded, directly or indirectly, to a lipid-soluble antioxidant and further relates to reduced sulfhydryl derivatives of such compounds.

The present invention also relates to compounds which include a water-soluble antioxidant that is covalently bonded, directly or indirectly, to a lipid-soluble antioxidant.

### BRIEF DESCRIPTION OF THE DRAWINGS

FIG. 1 is a graph showing the effect of a compound in accordance with the present invention on the inhibition of maliondialdehyde production in Fe²⁺-induced stimulation of lipid peroxidation assay.

FIG. 2 is a graph showing the effect of α-tocopherol on the inhibition of maliondialdehyde production in Fe²+-induced stimulation of lipid peroxidation assay.

### DETAILED DESCRIPTION OF THE INVENTION

As used herein, “alkyl” is meant to include linear alkyls, branched alkyls, and cycloalkyls, each of which can be substituted or unsubstituted. “Alkyl” is also meant to include lower linear alkyls (e.g., C1-C6 linear alkyls), such as methyl, ethyl, n-propyl, n-butyl, n-pentyl, and n-hexyl; lower branched alkyls (e.g., C3-C8 branched alkyls), such as isopropyl, t-butyl, 1-methylpropyl, 2-methylpropyl, 1-methylbutyl, 2-methylbutyl, 3-methylbutyl, 1,2-dimethylpropyl, 1,1-dimethylpropyl, 2,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 2-methyl-2-ethylpropyl, 2-methyl-1-ethylpropyl, and the like; and lower cycloalkyls (e.g., C3-C8 cycloalkyls), such as cyclopropyl, cyclobutyl, cyclopentyl, cyclohexyl, and the like. “Alkyl”, as used herein, is meant to include unsubstituted alkyls, such as those set forth above, in which no atoms other than carbon and hydrogen are present. “Alkyl”, as used herein, is also meant to include substituted alkyls. Suitable substituents include aryl groups (which may themselves be substituted), heterocyclic rings (saturated or unsaturated and optionally substituted), hydroxy groups, alkoxy groups (which is meant to include aryloxy groups (e.g., phenoxy groups)), amine groups (unsubstituted, monosubstituted, or disubstituted, e.g., with aryl or alkyl groups), carboxylic acid groups, carboxylic acid derivatives (e.g., carboxylic acid esters, amides, etc.), sulfonic acid groups, halogen atoms (e.g., Cl, Br, and I), and the like. Further, alkyl groups bearing one or more alkenyl or alkynyl substituents (e.g., a methyl group itself substituted with a prop-1-en-1-yl group to produce a but-2-en-1-yl substituent) is meant to be included in the meaning of “alkyl”.

As used herein, “alkylene” refers to a bivalent alkyl group, where alkyl has the meaning given above. Linear, branched, and cyclic alkylenes, as well as examples thereof, are defined in similar fashion with reference to their corresponding alkyl group. Examples of alkylenes include eth-1,1-diyl (i.e., —CH(CH₃)—), eth-1,2-diyl (i.e., —CH₂CH₂—), prop-1,1-diyl (i.e., —CH(CH₂CH₃)—), prop-1,2-diyl (i.e.,—CH₂—CH(CH₃)—), prop-1,3-diyl (i.e., —CH₂CH₂CH₂—), prop-2,2-diyl (e.g. —C(CH₃)₂—), cycloprop-1,1-diyl, cycloprop-1,2-diyl, cyclopent-1,1-diyl, cyclopent-1,2-diyl, cyclopent-1,3-diyl, cyclohex-1,1-diyl, cyclohex-1,2-diyl, cyclohex-1,3-diyl, cyclohex-1,4-diyl, but-2-en-1,1-diyl, cyclohex-1,3-diyl, but-2-en-1,4-diyl, but-2-en-1,2-diyl, but-2-en-1,3-diyl, but-2-en-2,3-diyl. Also included in the meaning of the term “alkylene” are compounds having the formula —R′—R″—, where —R′ represents a linear or branched alkyl group and R″— represents a cycloalkyl group, such as moieties having the formula:

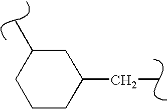

As used herein, “aryl” is meant to include aromatic rings, preferably having from 4 to 12 members, such as phenyl rings. These aromatic rings can optionally contain one or more heteroatoms (e.g., one or more of N, O, and S), and, thus, “aryl”, as used herein, is meant to include heteroaryl moieties, such as pyridyl rings and furanyl rings. The aromatic rings can be optionally substituted. “Aryl” is also meant to include aromatic rings to which are fused one or more other aryl rings or non-aryl rings. For example, naphthyl groups, benzimidazole groups, and 5,6,7,8-tetrahydro-2-naphthyl groups (each of which can be optionally substituted) are aryl groups for the purposes of the present application. As indicated above, the aryl rings can be optionally substituted. Suitable substituents include alkyl groups (which can optionally be substituted), other aryl groups (which may themselves be substituted), heterocyclic rings (saturated or unsaturated), hydroxy groups, alkoxy groups (which is meant to include aryloxy groups (e.g., phenoxy groups)), amine groups (unsubstituted, monosubstituted, or disubstituted, e.g., with aryl or alkyl groups), carboxylic acid groups, carboxylic acid derivatives (e.g., carboxylic acid esters, amides, etc.), sulfonic acid groups, halogen atoms (e.g., Cl, Br, and I), and the like.

As used herein, “alkoxy” is meant to include groups having the formula —O—R, where R is an alkyl or aryl group. They include methoxy, ethoxy, propoxy, phenoxy, 4-methylphenoxy, and the like.

In choosing suitable substituents, care should be taken not to employ substituents which adversely affect the anti-oxidant or anti-free radical properties of the compounds of the present invention.

The present invention relates to a compound which includes a cyclic or a cyclic disulfide that is covalently bonded, directly or indirectly, to a lipid-soluble antioxidant. The present invention further relates to a reduced sulfhydryl derivative of such a compound.

As used herein “compound” is meant to include non-ionic, adduct-free compounds, as well as salts (e.g., pharmaceutically acceptable salts) of such compounds and adducts of such compounds (e.g., compounds which further include x molecules of salvation or crystallization, such as ·xH₂O, ·xEtOH). Where a compound of the present invention is illustrated with a chemical formula, it is to be understood that the chemical formula is meant to include adducts thereof. Likewise, where the present application refers to “reduced sulfhydryl derivatives”, such is meant to include non-ionic, adduct-free reduced sulfhydryl derivatives, as well as salts (e.g., pharmaceutically acceptable salts) of such reduced sulfhydryl derivatives and adducts of such reduced sulfhydryl derivatives.

As used herein, “cyclic disulfide” means a ring or ring system which includes, within the ring or ring system, two sulfur atoms which are bonded to one another via a S—S bond.

As used herein, “acyclic disulfide” means two sulfur atoms which are bonded to one another via a S—S bond, which S—S bond is not part of a ring or ring system.

As used herein, “a reduced sulfhydryl derivative” of a compound which contains two sulfur atoms which are bonded to one another via a S—S bond refers to the compound in which the S—S bond is broken and each of the two sulfur atoms is bonded to a hydrogen.

Illustrative acyclic disulfides include those having the formula:

E—S—S—

[symbol]

where E is a substituted or unsubstituted alkyl or a ring (e.g., an aromatic ring or a non-aromatic ring). Useful acyclic disulfides include those which have antioxidant activity similar to (e.g., from 50% to 200%) that of glutathione disulfide (GSSG) or glutathione (GSH).

Illustrative cyclic disulfides include those having the formula:

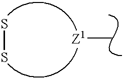

where Z¹ represents the atoms necessary to complete a ring, such as a 4-8-membered ring (e.g., a 4-, 5-, 6-, 7-, or 8-membered ring). The ring can contain, one or more additional heteroatoms (i.e., in addition to the two sulfur atoms), such as O, S, N, or all of the remaining ring atoms can be carbon. The ring can be saturated, or it can be unsaturated. For example, Z¹ can represent a substituted or unsubstituted alkylene moiety, such as a substituted or unsubstituted C2-C6 alkylene moiety and/or a substituted or unsubstituted C3-C5 alkylene moiety.

Suitable cyclic disulfides also include those having the formula:

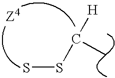

where Z⁴ represents a substituted or unsubstituted C2-C5 alkylene moiety, such as an unsubstituted C2-C5 alkylene moiety or a C2-C5 alkylene moiety bearing only one or more alkyl substituents. Illustratively, Z⁴ can represent an unsubstituted C2-C5 alkylene moiety, such as a —CH₂CH₂— moiety or a —CH₂CH₂CH₂— moiety.

Suitable cyclic disulfides also include those having the formula:

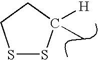

as well as those which have antioxidant activity similar to (e.g., from 50% to 200%) that of lipoic acid (LA) or dihydrolipoic acid (DHLA).

As indicated above, the compounds of the present invention further include a lipid-soluble antioxidant.

As used in this context, “antioxidant” is meant to refer to materials which (i) are capable of inhibiting (e.g., a by between about 10% and 100%, such as a by between about 20% and 100%, by between about 30% and 100%, by between about 40% and 100%, by between about 50% and 100%, by between about 60% and 100%, by between about 70% and 100%, by between about 80% and 100%, and/or by between about 90% and 100%) the activity of oxidants, particularly in biological environments, as measured, for example, by using standard assays for antioxidant activity, such as the inhibition of ferrous ion-stimulated formation of maliondialdehyde in microsomes or liposomes; or (ii) are at least about 50% (e.g., at least about 60%, at least about 70%, at least about 80%, at least about 90%, at least about 100%, at least about 110%, and/or at least about 120%) as effective in inhibiting the activity of oxidants as α-tocopherol, as measured, for example, by using standard assays for antioxidant activity, such as the inhibition of ferrous ion-stimulated formation of maliondialdehyde in microsomes or liposomes.

As used herein, an antioxidant is to be deemed to be “lipid-soluble” if (i) its lipid solubility is at least about 50% that of α-tocopherol (e.g., as in the case where the lipid-soluble antioxidant has a lipid solubility of at least about 60% that of α-tocopherol, at least about 70% that of α-tocopherol, at least about 80% that of α-tocopherol, at least about 90% that of α-tocopherol, at least about 100% that of α-tocopherol, and/or greater than that of α-tocopherol) or (ii) its water-octanol partition coefficient, P (where P=[antioxidant]_{octanol}/[antioxidant]_{water}), is greater than about 3.5, such as greater than about 4, greater than about 4.5, greater than about 5, greater than about 5.5, greater than about 6, greater than about 6.5, greater than about 7, greater than about 7.5, and/or greater than about 8.

Illustrative lipid-soluble antioxidants suitable for use in the compounds of the present invention include those which contain a tocopherol ring system which is substituted with at least one lipophilic moiety and which is otherwise substituted or unsubstituted. As used herein, “tocopherol ring system” is meant to refer to a substituted or unsubstituted 3,4-dihydrobenzopyran ring system.

As used herein, “lipophilic moiety” is meant to include, for example, hydrocarbons, such as unsubstituted alkyl groups having from 5 to 25 carbon atoms (e.g., hexyl, dodecyl, or 3,7,11-trimethyldodecyl groups), substituted alkyl groups (e.g., a benzyl or phenylethyl groups), homocyclic rings, homocyclic ring systems, heterocyclic rings, heterocyclic ring systems, aromatic hydrocarbons, lipophilic bicycloalkanes (e.g., adamantyl groups), and the like.

For example, the lipid-soluble antioxidant can be one having the formula:

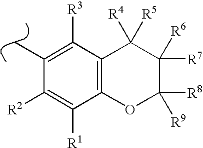

where R¹-R⁹ are independently selected from the group consisting of hydrogen, a substituted or unsubstituted alkyl, a substituted or unsubstituted 4-8 membered homocyclic ring, a substituted or unsubstituted 4-8 membered heterocyclic ring, a hydroxy group, a substituted or unsubstituted alkoxy group, a substituted or unsubstituted amine group, a halogen, a carboxylic acid group, a carboxylic acid ester group, and a carboxylic acid amide group, provided that at least one of R¹-R⁹ is a lipophilic moiety; such as in the case where (i) R¹-R⁹are independently selected from the group consisting of hydrogen and substituted or unsubstituted alkyls, provided that at least one of R¹-R⁹ is a lipophilic moiety and/or (ii) R¹-R³ and R⁹ are independently selected from the group consisting of hydrogen, substituted alkyls, and unsubstituted alkyls, one of R⁴-R⁸ is a substituted or unsubstituted lipophilic alkyl or aryl, and the remaining of R⁴-R⁸ are hydrogen and/or (iii) R¹-R³ and R⁹ are independently selected from the group consisting of hydrogen and a methyl group, one of R⁴-R⁸ is a substituted or unsubstituted lipophilic alkyl or aryl, and the remaining of R⁴-R⁸ are hydrogen and/or (iv) R¹-R³ and R⁹ are independently selected from the group consisting of hydrogen and a methyl group, each of R⁴-R⁷ is hydrogen, and R⁸ is a substituted or unsubstituted lipophilic alkyl and/or (v) each of R¹-R³ and R⁹ is a methyl group, one of R⁴-R⁸ is a substituted or unsubstituted lipophilic alkyl, and the remaining of R⁴-R⁸ are hydrogen and/or (vi) each of R¹-R³ and R⁹ is a methyl group, each of R⁴-R⁷ is hydrogen, and R⁸ is a substituted or unsubstituted lipophilic alkyl.

As indicated above, the cyclic or acyclic disulfide is covalently bonded, directly or indirectly, to the lipid-soluble antioxidant. For purposes of the present invention, cyclic or acyclic disulfide is to be deemed as being “covalently bonded, directly or indirectly” to a lipid-soluble antioxidant (i) if there is a direct covalent bond between the cyclic or acyclic disulfide and the lipid-soluble antioxidant or (ii) if the cyclic or acyclic disulfide and the lipid-soluble antioxidant are each covalently bonded to a bridging group, the atoms of which bridging group are covalently bonded to one another. Illustratively, the bridging group can have the formula:

—Z⁵—Z⁶—

where Z⁵ represents a substituted or unsubstituted C1-C8 alkylene moiety and Z⁶ represents an ester, an amide, a carbamate, a carbonate, an imine, a urea, or an enol ether functional group or linkage or another functional group of linkage which is susceptible to metabolic cleavage in vivo (e.g., by hydrolysis, by reduction, etc.); such as where Z⁵ represents a substituted or unsubstituted C3-C5 alkylene moiety and Z⁶ represents an ester functional group; and/or such as where Z⁵ represents an unsubstituted C3-C5 alkylene moiety and Z⁶ represents an ester functional group; and/or such as where Z⁵ represents a —CH₂CH₂CH₂— moiety, a —CH₂CH₂CH₂CH₂— moiety, or a —CH₂CH₂CH₂CH₂CH₂— moiety and Z⁶ represents an ester functional group. Illustrative ester linkages include those represented by the formula —C(O)—O—; illustrative amide linkages include those represented by the formula —C(O)—N(R¹⁰)—; illustrative carbamate linkages include those represented by the formula —N(R¹⁰)—C(O)—O—; illustrative carbonate linkages include those represented by the formula —O—C(O)—O—; illustrative imine linkages include those represented by the formula —C(R¹⁰)=N—; illustrative urea linkages include those represented by the formula —NH—C(O)—NH—; and illustrative enol ether linkages include those represented by the formula ═CR¹⁰—O—; where, in each of the above formulae, R¹⁰ can be hydrogen, substituted or unsubstituted alkyl, or substituted or unsubstituted aryl.

For example, compounds of the present invention include those having the formula:

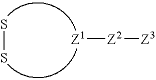

where Z¹ represents the atoms necessary to complete a ring, such as a 4-8-membered ring (e.g., where Z¹ represents a substituted or unsubstituted C2-C6 alkylene moiety); Z² represents a bridging moiety; and Z³ represents the lipid-soluble antioxidant; and the present invention further relates to reduced sulfhydryl derivatives of such compounds.

Illustratively, Z¹ can be a substituted or unsubstituted C3-C5 alkylene moiety; and/or Z¹, together with the S—S moiety to which it is bonded, can have the formula:

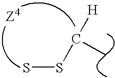

where Z⁴ represents a substituted or unsubstituted C2-C5 alkylene moiety (e.g., an unsubstituted C2-C5 alkylene moiety); and/or Z¹, together with the S—S moiety to which it is bonded, can have the formula:

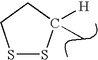

It should be noted that, in the case where cyclic disulfides are employed, the point of attachment to the cyclic disulfide is not particularly critical. For example, in the case where where Z¹ is a substituted or unsubstituted C3 alkylene moiety, another suitable cyclic disulfide is one having the following formula:

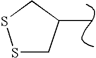

where, which cyclic disulfide can be unsubstituted or substituted with one or more substituents.

Illustratively, Z³ can represent a tocopherol ring system which is substituted with at least one lipophilic moiety and which is otherwise substituted or unsubstituted, for example, as in the case where Z³ has the formula:

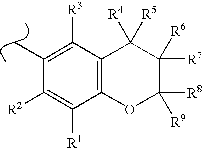

where R¹-R⁹ have any of the meanings set forth above. Examples of compounds of the present invention in which Z³ represents a tocopherol ring system include those compounds in which Z³ represents an α-tocopherol moiety, a β-tocopherol moiety, a γ-tocopherol moiety, a δ-tocopherol moiety, a ζ₁-tocopherol moiety, a ζ₂-tocopherol moiety, a η-tocopherol moiety, or a tocol moiety where the α-tocopherol moiety, β-tocopherol moiety, γ-tocopherol moiety, δ-tocopherol moiety, ζ₁-tocopherol moiety, ζ₂-tocopherol moiety, η-tocopherol moiety, or tocol moiety is covalently bonded to Z² via its hydroxyl carbon (i.e., via the aromatic carbon para to the ring system's oxygen atom). The present invention further relates to reduced sulfhydryl derivatives of such compounds.

Illustratively, the bridging group, Z², can have the formula:

—Z⁵—Z⁶—

where Z⁵ represents a substituted or unsubstituted C1-C8 alkylene moiety and Z⁶ represents an ester, an amide, a carbamate, a carbonate, an imine, a urea, or an enol ether functional group or linkage, for example, as further described hereinabove.

As a further example, compounds of the present invention include those having the formula:

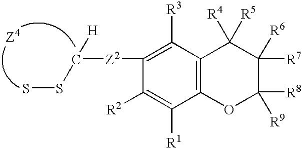

where Z², Z⁴, and R¹-R⁹ have the meanings set forth hereinabove; and the present invention further relates to reduced sulfhydryl derivatives of such compounds. More particularly, compounds of the present invention include those having the following formulae:

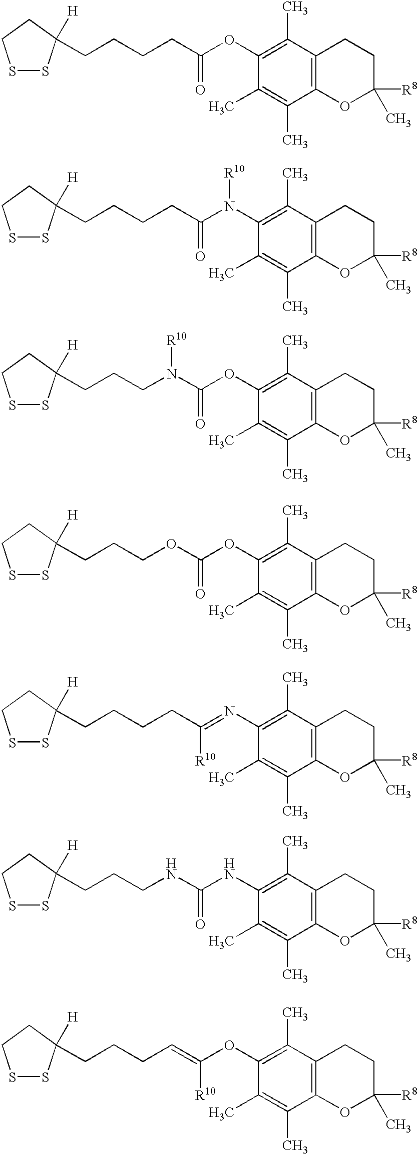

where R⁸ is a lipophilic moiety (e.g., an unsubstituted lipophilic alkyl group) and where R¹⁰ is selected from the group consisting of hydrogen, substituted or unsubstituted alkyl, and substituted or unsubstituted aryl; and the present invention further relates to reduced sulfhydryl derivatives of such compounds.

As one skilled in the art will appreciate, the compounds of the present invention (and their counterpart reduced sulfhydryl derivatives) may include one or more chiral carbon atoms. The structures set forth above, which do not specify the stereochemistry of such chiral centers, are meant to include all combinations of optical isomers, including racemic and non-racemic mixtures. The structures set forth above, which do not specify the stereochemistry of such chiral centers, are also meant to include optically pure compounds and reduced sulfhydryl derivatives of the present invention. Examples of such optically pure compounds of the present invention include those having the following formulae:

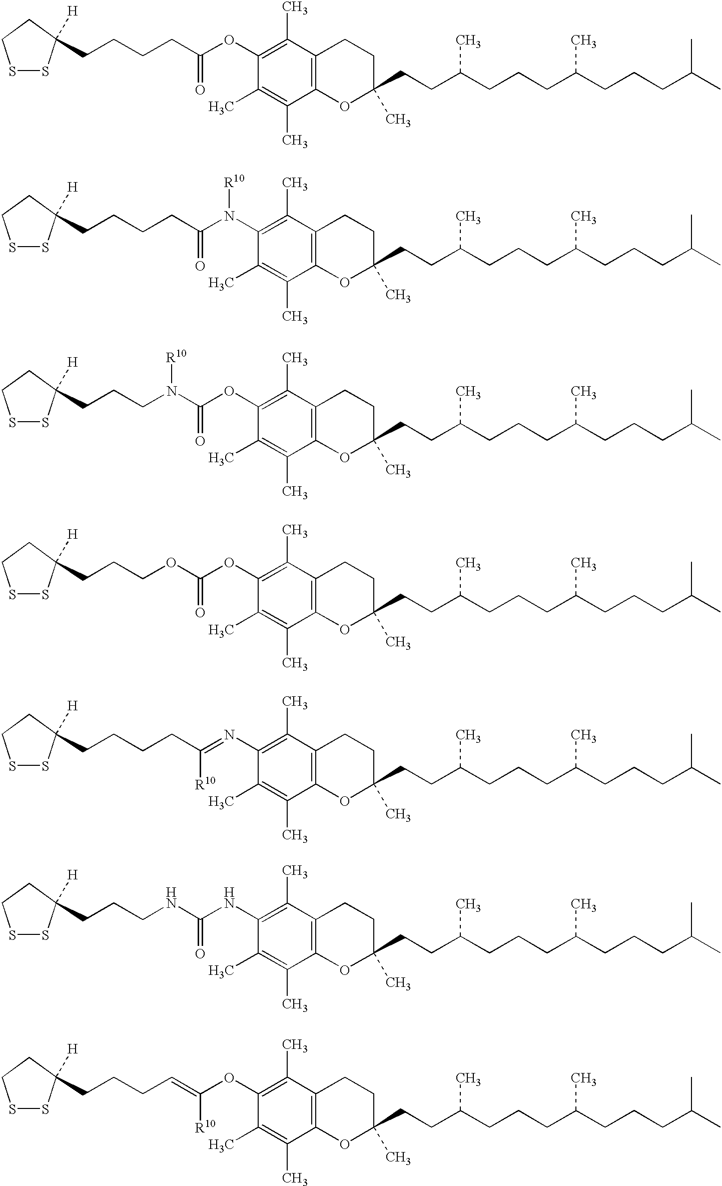

where R¹⁰ is a hydrogen, a substituted or unsubstituted alkyl, or a substituted or unsubstituted aryl. Examples of optically pure reduced sulfhydryl derivatives of the present invention include reduced sulfhydryl derivatives of the compounds represented by these formulae.

Where acyclic disulfides are employed, compounds of the present invention include those having the formula:

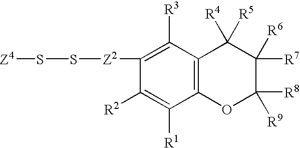

where Z² and R¹-R⁹ have the meanings set forth hereinabove; where Z⁴ represents a substituted or unsubstituted alkyl or aryl; and the present invention further relates to reduced sulfhydryl derivatives of such compounds. Illustratively, Z⁴ can represent a second lipid-soluble antioxidant covalently bonded, directly or indirectly to the disulfide's sulfur atom, such as in the case where Z⁴ is represented by a moiety having the formula:

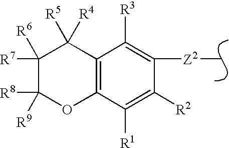

where Z² and R¹-R⁹ have the meanings set forth hereinabove.

The compounds of the present invention and/or their reduced sulfhydryl derivatives can be additionally of alternatively characterized in terms of their lipid solubility. For example, suitable compounds and reduced sulfhydryl derivatives thereof include those which have water-octanol partition coefficients, P (where P=[antioxidant]_{octanol}/[antioxidant]_{water}), is greater than about 3.5, such as greater than about 4, greater than about 4.5, greater than about 5, greater than about 5.5, greater than about 6, greater than about 6.5, greater than about 7, greater than about 7.5, greater than about 8.

The compounds of the present invention can be prepared by any suitable method. Generally, this involves reacting a cyclic or acyclic disulfide with a lipid-soluble antioxidant under conditions effective to covalently bond, directly or indirectly, the cyclic or acyclic disulfide to the lipid-soluble antioxidant. Where indirect bonding is desired, one end of a bridging moiety can be covalently coupled to the cyclic or acyclic disulfide prior to reacting the other end of the bridging moiety with the lipid-soluble antioxidant. Alternatively, one end of a bridging moiety can be covalently coupled to the lipid-soluble antioxidant prior to reacting the other end of the bridging moiety with the cyclic or acyclic disulfide. Still alternatively, the lipid-soluble antioxidant, the cyclic or acyclic disulfide, and the bridging moiety can be reacted together in a single mixture (e.g., simultaneously). Of course, the nature of the starting materials will, in part, determine the order of reaction. For example, where each of the cyclic or acyclic disulfide and the lipid-soluble antioxidant includes a reactive hydroxyl function (e.g., a phenolic OH group or a alcoholic OH group), the reaction can be conveniently carried out in a single step by reacting the cyclic or acyclic disulfide and the lipid-soluble antioxidant with a bridging moiety having carboxylic acid groups at both ends under conditions conducive for ester-formation. Alternatively, the reaction can be carried out stepwise, for example, by first reacting one of the bridging moiety's carboxylic acid groups with the lipid-soluble antioxidant's reactive hydroxyl group; optionally separating and/or purifying the resulting intermediate compound; and then reacting the intermediate compound with the cyclic or acyclic disulfide's reactive hydroxyl group. As one skilled in the art will note, where stepwise synthesis is carried out, it may be desirable to protect one of the bridging moiety's carboxylic acid groups prior to carrying out the first step of the reaction, and then to de-protect the carboxylic acid group prior to carrying out the second step of the reaction. Other analogous synthetic strategies can be used to covalently bond cyclic or acyclic disulfide and lipid-soluble antioxidants via amide, carbamate, carbonate, imine, urea, and enol ether linkages. Details regarding reaction conditions and starting materials suitable for formation of such linkages can be found in, for example, Morrison et al., Organic Chemistry, 3rd ed., Boston, Mass.: Allyn & Bacon, Inc. (1973) and Kemp et al., Organic Chemistry, New York: Worth Publishers, Inc. (1980), which are hereby incorporated by reference.

For example, compounds of the present invention which have the formula:

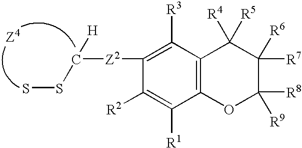

in which Z² is —Z⁵—C(O)—O—, can be prepared by providing a benzopyran having the formula:

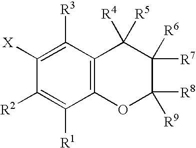

where X represents a hydroxy group or a protected hydroxy group. As used herein, “protected hydroxy group” is meant to refer to groups having the formula O⁻M⁺, where M⁺ is a cation (e.g., Na⁺, Li⁺, [N(CH₂CH₃)₄]⁺), and to other functional groups which can be readily converted to a hydroxy group.

The method further includes converting the benzopyran with a disulfide having the formula:

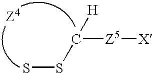

where X′ represents a carboxylic acid group or a protected carboxylic acid group. As used herein, “protected carboxylic acid group” is meant to refer to carboxylic acid salts (e.g., a sodium salt, a lithium salt, a tetraalkylammonium salt, etc.), carboxylic acid esters, and other functional groups which can be readily converted to a carboxylic acid group.

The conversion of the benzopyran with the disulfide can be carried out, for example, by dissolving or suspending the benzopyran in a suitable solvent (e.g., a chlorinated hydrocarbon, such as methylene chloride), dissolving or suspending the disulfide in the same or a separate solvent (e.g., a chlorinated hydrocarbon, such as methylene chloride), and contacting the benzopyran solution or suspension with the disulfide solution or suspension, preferably with stirring. Typically, the reaction is carried out using a benzopyran:disulfide mole ratio of from 0.5:1 to 2:1, preferably from 0.8:1 to 1.2:1, more preferably about 1:1. Preferably, a suitable dehydration agent and/or other means for removing the water formed as a consequence of the reaction are employed. Illustratively, dicyclohexylcarbodiimide can be used as a dehydration agent, and it is preferred that a large mole excess (e.g., a 3-4-fold mole excess) of dehydration agent be employed. A suitable catalyst (e.g., a Lewis base, such as 4-dimethylaminopyridine) can also be used advantageously. Typically, the reaction is carried out from about 10° C. to about 50° C. (e.g., at room temperature) for from about 2 hours to about 4 days (e.g., preferably from about 12 hours to about 48 hours, such as about 24 hours). The progress of the reaction can be monitored using standard methods, such as by periodically removing aliquots from the reaction mixture and analyzing them chromatographically (e.g., by thin layer chromatography).

The resulting product can optionally be purified by a variety of methods, such as by column chromatography, HPLC, recrystallization, and the like.

Of course, the aforementioned method can include other steps. For example, in the case where X is a group having the formula O⁻M⁺, the benzopyran can be first reacted with an acid to convert the O⁻M⁺ group to an OH group, and the resulting OH-containing benzopyran can then be reacted with the disulfide, for example as described above. As an additional example, in the case where X′ is a group having the formula COO⁻M⁺, the disulfide can be first reacted with an acid to convert the COO⁻M⁺ group to an COOH group, and the resulting COOH— containing disulfide can then be reacted with the benzopyran, for example as described above.

Benzopyran starting materials suitable for use in the practice of the method of the present invention can be obtained commercially, or they can be prepared from commercially available materials by methods known to those skilled in the art. Illustratively, α-tocopherol can be obtained from Aldrich Chemical Co., St. Louis, Mo., or it can be prepared by the methods described in Karrer et al., Helv. Chim. Acta, 21:520ff (1938); Bergel et al., J. Chem. Soc., pp. 1382ff (1938); Smith et al., Science, 88:37ff (1938); Smith et al., J. Am. Chem. Soc., 65:1276ff (1943); Cohen et al., Helv. Chim. Acta, 61:837ff (1978); Cohen et al., J. Am. Chem. Soc., 101:6710ff (1979); Barner et al., Helv. Chim. Acta, 62:2384ff (1979); and Heathcock et al., Tett. Lett., 23:2825 (1982), which are hereby incorporated by reference. Each of β-tocopherol and γ-tocopherol can be obtained from the fractional crystallization of allophanates, for example, by the methods described in Emerson et al., Science, 83:421ff (1936); Emerson et al., J. Biol. Chem., 113:319ff (1936); and Baxter et al., J. Am. Chem Soc., 65:918ff (1943), which are hereby incorporated by reference. δ-Tocopherol can be isolated from soybean oil, for example, as described in Stern et al., J. Am. Chem. Soc., 69:869ff (1947), which is hereby incorporated by reference, or it can be prepared, for example, by the methods described in Green et al., J. Chem. Soc., pp. 3374ff (1959) and British Patent No. 900,085 to Hoffmann-La Roche, which are hereby incorporated by reference. ε-Tocopherol can be isolated from wheat germ oil and from bran, for example, as described in Eggitt et al., J. Sci. Food Agr., 4:569ff (1953) and Eggitt et al., J. Sci. Food Agr., 6:689ff (1955) which are hereby incorporated by reference, or it can be prepared, for example, by the methods described in Schudel et al., Helv. Chim. Acta, 46:2517ff (1963), which is hereby incorporated by reference. ζ₁-Tocopherol can be isolated from wheat bran, for example, as described in Green et al., J. Sci. Food Aqr., 6:274ff (1955) and Green et al., Chem. & Ind. (London), pp. 73ff (1960) which are hereby incorporated by reference, or it can be prepared, for example, by the methods described in Schudel et al., Helv. Chim. Acta, 46:2517ff (1963), which is hereby incorporated by reference. ζ₂-Tocopherol can be isolated from rice, for example, as described in Green et al., Nature, 177:86ff (1956), which is hereby incorporated by reference, or it can be prepared, for example, by the methods described in Karrer et al., Helv. Chim. Acta, 21:1234ff (1938); Bergel et al., J. Chem. Soc., pp. 1382ff (1938); and McHale et al., J. Chem. Soc., pp. 1600ff (1958), which are hereby incorporated by reference. η-Tocopherol can be isolated from rice, for example, as described in Green et al., Nature, 177:86ff (1956), which is hereby incorporated by reference, or it can be prepared, for example, by the methods described in McHale et al., J. Chem. Soc., pp. 1600ff (1958); Green et al., J. Chem. Soc., pp. 3374ff (1959); and Marcinkiewicz et al., J. Chem. Soc., pp. 3377ff (1959), which are hereby incorporated by reference. Tocol can be prepared, for example, by the methods described in Pendse et al., Helv. Chim. Acta, 40:1837ff (1957), which is hereby incorporated by reference. Other benzopyran starting materials can be prepared by routine modifications to the side chains of tocol and/or α-, β- γ-, δ-, ζ₁-, ζ₂-, and/or η-tocopherol or by routine modifications to the above-cited synthetic procedures for the preparation of tocol and α-, β- γ-, δ-, ζ₁, ζ₂-, and η-tocopherol.

Disulfide starting materials suitable for use in the practice of the method of the present invention can be obtained commercially, or they can be prepared from commercially available materials by methods known to those skilled in the art. Illustratively, lipoic acid (thioctic acid) can be obtained commercially from Aldrich Chemical Co., St. Louis, Mo., or it can be prepared using the methods set forth, for example, in Bullock et al., J. Am. Chem Soc., 74:1868ff (1952); Bullock et al., J. Am. Chem Soc., 74:3455ff (1952); Hornberger et al., J. Am. Chem Soc., 74:2382ff (1952); U.S. Pat. No. 2,980,716 to Reed; U.S. Pat. No. 3,049,549 to Reed; Lewis et al., J. Chem. Soc., pp. 4263ff (1962); U.S. Pat. No. 3,223,712 to Ose et al.; and Tsuji et al., J. Org. Chem., 43:3606ff (1978), which are hereby incorporated by reference. Other disulfide starting materials can be prepared by routine modifications to the above-cited synthetic procedures for the preparation of lipoic acid.

Further details regarding certain aspects of the synthesis of compounds of the present invention can be found, for example, in Saah et al., “Design, Synthesis, and Pharmacokinetic Evaluation of a Chemical Delivery System for Drug Targeting to Lung Tissue,” J. Pharm. Sci., 85:496-504 (1996), which is hereby incorporated by reference.

As indicated above the present invention also relates to the reduced sulfhydryl derivatives of the aforementioned compounds. Such reduced sulfhydryl derivatives can be prepared, for example, from the compounds of the present invention by contacting the compounds of the present invention with a suitable reducing agent, such as Zn/H⁺.

As a further illustration, compounds of the present invention containing bridging groups bearing amide linkages can be prepared in accordance with the following Scheme I:

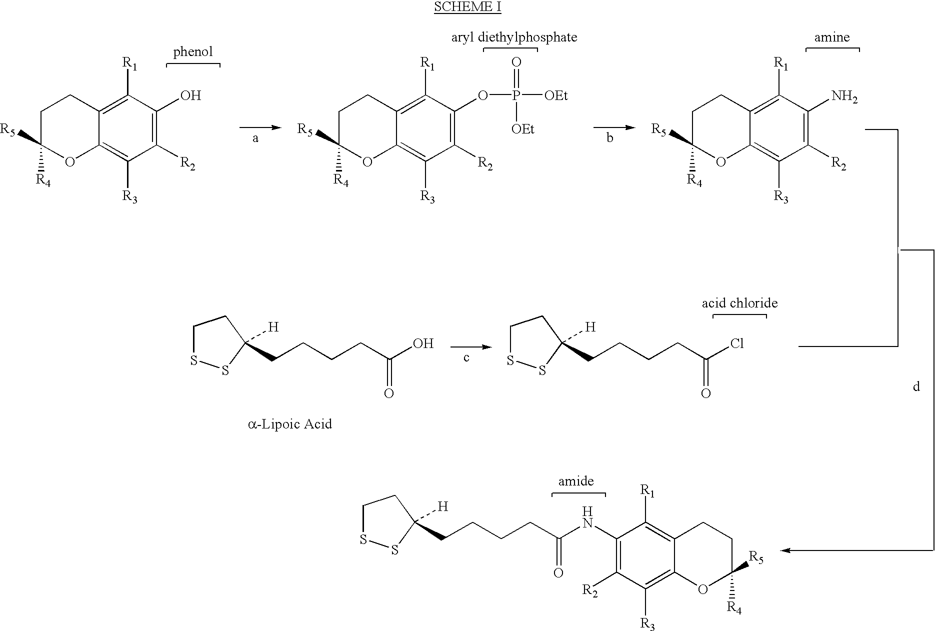

Step “a” can be carried out with NaOH and (EtO)₂POCl, for example, as described in Rossi and Bunnett, J. Org. Chem., 37:3570ff (1972) (“Rossi”), which is hereby incorporated by reference Step “b” can be carried out with KNH₂ and NH₃, for example, as described in Rossi and in Scherrer and Beatty, J. Org. Chem., 37:168ff (1972) (“Scherrer”), which are hereby incorporated by reference. Step “c” can be carried out with SOCl₂ using, for example, the procedure described in Ansell, pp. 35-68 in Patai, The Chemistry of Acyl Halides, New York: Interscience (1972), which is hereby incorporated by reference. Step “d” can be carried out using, for example, the procedures described in Challis and Challis, pp. 731-857 in Zabicky, The Chemistry of Amides, New York: Interscience (1970), which is hereby incorporated by reference. In the method depicted in Scheme I, each of the starting materials (e.g., commercially available α-lipoic acid and α-tocopherol) undergo functional group transformation prior to reaction with one another. More particularly, Scheme I shows (i) the carboxylic acid functionality of the α-lipoic acid being converted to an acid chloride with thionyl chloride (Step “c”) and (ii) the phenol being converted to an amine in a two-step reaction sequence (Steps “a” and “b”). Reaction of the amine functional group of the modified benzopyran (e.g., α-tocopherol) with the acyl chloride of the modified lipoic acid (Step “d”) produces the amide analog.

Compounds of the present invention containing bridging groups bearing carbamate linkages can be prepared in accordance with the following Scheme II:

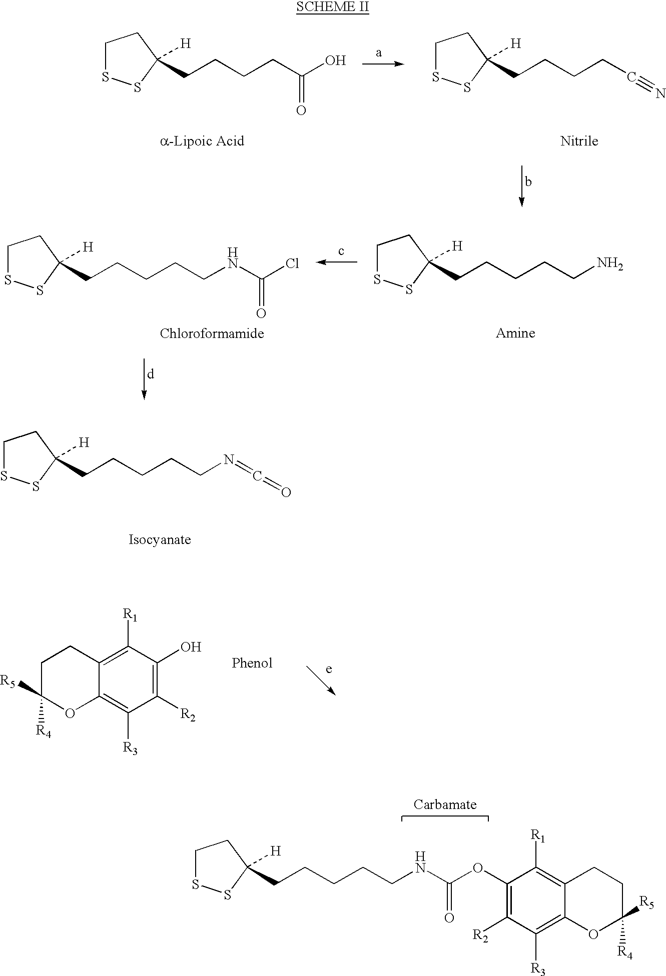

Step “a” can be carried out with BrCN, for example, as described in Barltrop et al., J. Chem. Soc., 3085ff (1961), which is hereby incorporated by reference. Step “b” can be carried out with LiAlH₄ or H₂ in the presence of a suitable catalyst (e.g., Pt), for example, as described in Rabinowitz, pp. 307-340 in Rappoport, The Chemistry of the Cyano Group, New York: Interscience (1970), which is hereby incorporated by reference, followed by treatment with H₂O₂, for example, as described in Capozi and Modena, pp. 785-839 in Patai, The Chemistry of the Thiol Group, Part 2, New York: Wiley (1974) (“Capozi”), which is hereby incorporated by reference. Step “c” can be carried out with trichloromethylchloroformate (or another trihalomethylchloroformate, such as F₃COC(O)Cl), for example, as described in Kurita and Iwakura, Org. Synth., 59:195ff (1979) (“Kurita”) and in Patai, The Chemistry of Cyanates and Their Thiol Derivatives, Part 2, pp. 619-818 and 1003-1221, New York: Wiley (1977) (“Patai”), which are hereby incorporated by reference. Step “d”, the reaction of the chloroformamide with, e.g., α-tocopherol, can be carried out, for example, using the methods described in Satchell and Satchell, Chem. Soc. Rev., 4:231ff (1975) and Satchell and Satchell, Chem. Soc. Rev., 4:250ff (1975), which are hereby incorporated by reference. In the method illustrated in Scheme II, the carboxylic acid functionality of the α-lipoic acid is converted to an amine via a nitrile intermediate. The nitrile is prepared by treatment of the acid with cyanobromide (Step “a”). Reduction of the nitrile, e.g., with lithium aluminum hydride or via catalytic hydrogenation, generates the amine. Under certain conditions, the reducing agents may reduce the disulfide to the sulfhydryl derivative, in which case oxidation back to the disulfide can be accomplished with, for example, hydrogen peroxide (Step “b”). Upon reaction of the primary amine with trichloromethylchloroformate, the chloroformamide is generated (Step “c”)). Loss of HCl from the chloroformamide generates the isocyanate, as shown in Step “d”. The resulting isocyanate can then react with the phenolic functional group of, for example, α-tocopherol to give the carbamate product (Step “e”).

Compounds of the present invention containing bridging groups bearing carbonate linkages can be prepared in accordance with the following Scheme III:

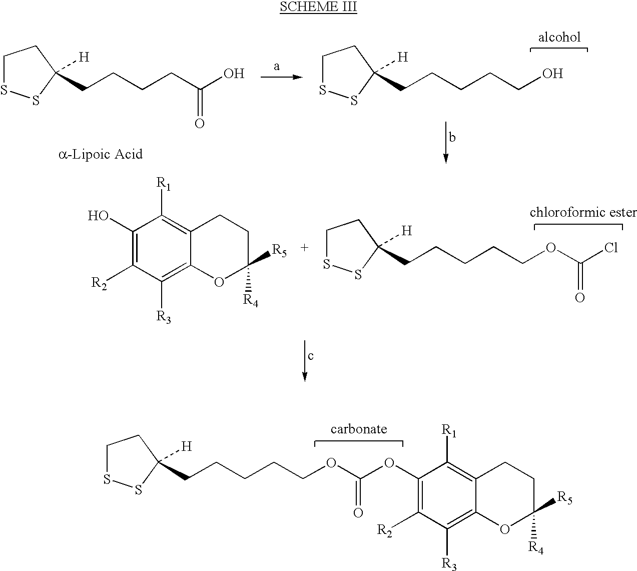

Step “a” can be carried out with LiAlH₄, for example, as described in House, Modern Synthetic Reactions, 2nd ed., Menlo Park, Calif.: W. A. Benjamin, p. 71 (1972) (“House”), which is hereby incorporated by reference, followed by treatment with H₂O₂, for example, as described in Capozi, which is hereby incorporated by reference. Step “b” can be carried out using trichloromethylchloroformate or phosgene, for example, as described in Kurita, in Patai, and in Matzner et al., Chem. Rev. 64:645-687 (1964), which are hereby incorporated by reference. Step “c”, the reaction of the chloroformic ester with, e.g., α-tocopherol, can be carried out, for example, using the methods described in Illi, Tetrahedron Lett., 2431 (1979). Alternatively, in Step “c”, the reaction of the chloroformic ester can be carried out with a phenoxide salt, for example, as described in Kaiser and Woodruff, J. Org. Chem., 35:1198ff (1970), which is hereby incorporated by reference. In the method illustrated in Scheme III, the carboxylic acid functionality of the α-lipoic acid is reduced, e.g., with LiAlH₄. Under certain conditions, the reducing conditions employed may reduce the disulfide to the sulfhydryl derivative, in which case oxidation back to the disulfide can be accomplished with, for example, hydrogen peroxide (Step “a”). The resulting alcohol can then be reacted with phosgene or trichloromethylchloro-formate to produce the chloroformic ester (Step “b”). Treatment of the chloroformic ester with a phenol (e.g., 60 -tocopherol) or a phenoxide salt (e.g., a phenoxide salt of 60 -tocopherol) provides the carbonate analog (Step “c”).

Compounds of the present invention containing bridging groups bearing imine linkages can be prepared in accordance with the following Scheme IV:

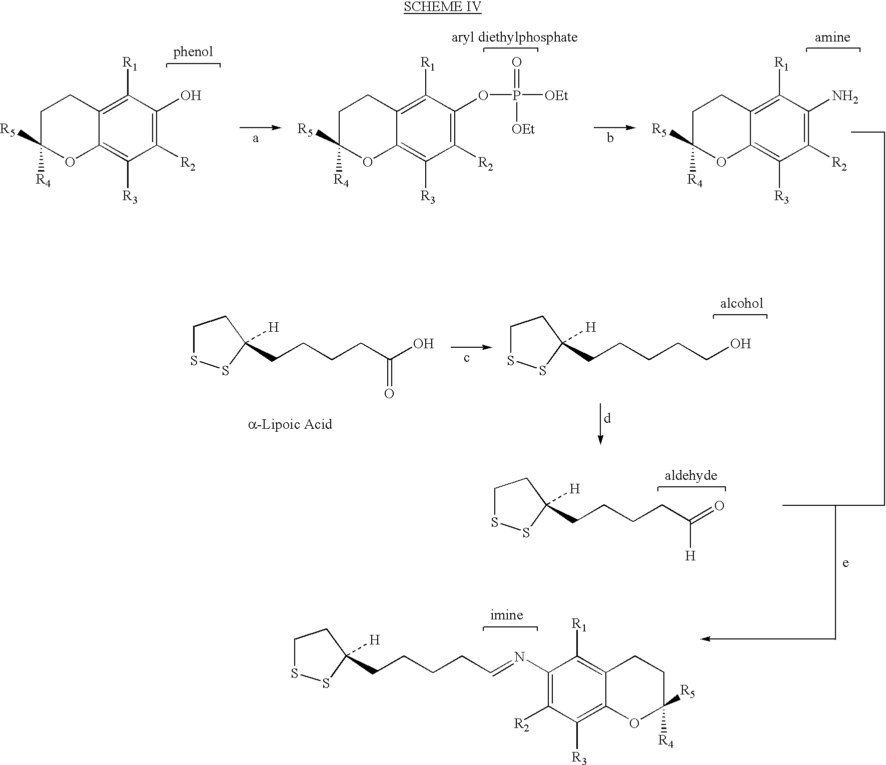

Step “a” can be carried out with NaOH and (EtO)₂POCl, for example, as described in Rossi, which is hereby incorporated by reference. Step “b” can be carried out with KNH₂ and NH₃, for example, as described in Rossi and in Scherrer, which are hereby incorporated by reference. Step “c” can be carried out with LiAlH₄, for example, as described in House, which is hereby incorporated by reference, followed by treatment with H₂O₂, for example, as described in Capozi, which is hereby incorporated by reference. Step “d” can be carried out using, for example, pyridium-chloro-chromate (“PCC”), using a procedure such as that described in Brown, Kilkarni, and Rao, Synthesis, 151 (1980), which is hereby incorporated by reference. Step “e” can be carried out, for example, using the procedures described in March, Advanced Organic Chemistry, 3rd ed., New York: John Wiley & Sons, pp. 796-797 (1985), which is hereby incorporated by reference. In the method depicted in Scheme IV, each of the starting materials (e.g., commercially available α-lipoic acid and α-tocopherol) undergo functional group transformation prior to reaction with one another. More particularly, Scheme IV shows the carboxylic acid functionality of the α-lipoic acid being converted to an alcohol (e.g., with LiAlH₄, followed, if necessary, with a peroxide treatment to restore the disulfide functionality) (Step “c”) . The alcohol is then oxidized to the aldehyde, e.g., using PCC (Step “d”). Scheme IV also shows the phenol being converted to an amine in a two-step reaction sequence (Steps “a” and “b”). Reaction of the amine functional group of the modified benzopyran (e.g., α-tocopherol) with the aldehyde of the modified lipoic acid (Step “e”) produces the imine analog.

Compounds of the present invention containing bridging groups bearing urea linkages can be prepared in accordance with the following Scheme V:

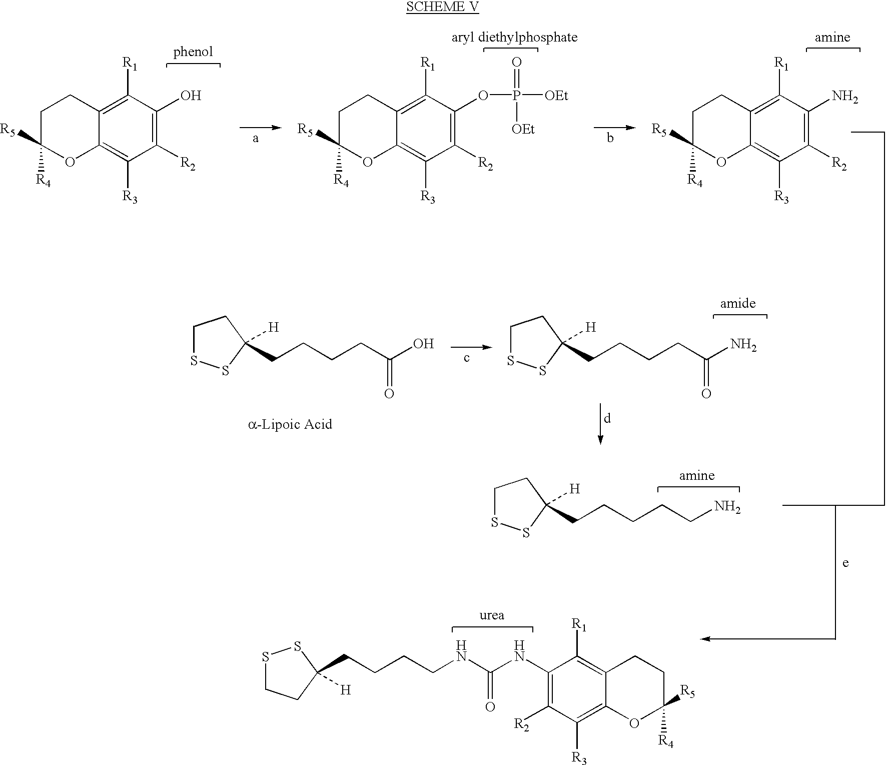

Step “a” can be carried out with NaOH and (EtO)₂POCl, for example, as described in Rossi, which is hereby incorporated by reference. Step “b” can be carried out with KNH₂ and NH₃, for example, as described in Rossi and in Scherrer, which are hereby incorporated by reference. Step “c” can be carried out with SOCl₂ followed by treatment with NH₃, for example, as described in Shriner et al., The Systematic Identification of Organic Compounds, 7th ed., New York: John Wiley & Sons, p. 309 (1997), which is hereby incorporated by reference. Step “d” can be carried out with LiAlH₄, for example, as described in House, which is hereby incorporated by reference, followed by treatment with H₂O₂, for example, as described in Capozi, which is hereby incorporated by reference. Step “e” can be carried out with trichloromethylchloroformate (or another trihalomethylchloroformate, such as F₃COC(O)Cl), for example, as described in Kurita and in Patai, which are hereby incorporated by reference. In the method depicted in Scheme V, each of the starting materials (e.g., commercially available α-lipoic acid and α-tocopherol) undergo functional group transformation prior to reaction with one another. More particularly, Scheme IV shows the carboxylic acid functionality of the a-lipoic acid being converted to an amide, for example, with thionyl chloride and ammonia (Step “c”). The amide is reduced to an amine (e.g., with LiAlH₄, followed, if necessary, with a peroxide treatment to restore the disulfide functionality) (Step “d”). Scheme V also shows the phenol being converted to an amine in a two-step reaction sequence (Steps “a” and “b”). Reaction of the amine functional group of the modified benzopyran (e.g., α-tocopherol) with, for example, trichloromethylchloroformate, followed by treatment of the resulting isocyanate with the amine of the modified lipoic acid (Step “e”) produces the urea analog.

Compounds of the present invention containing bridging groups bearing enol ether linkages can be prepared in accordance with the following Scheme VI:

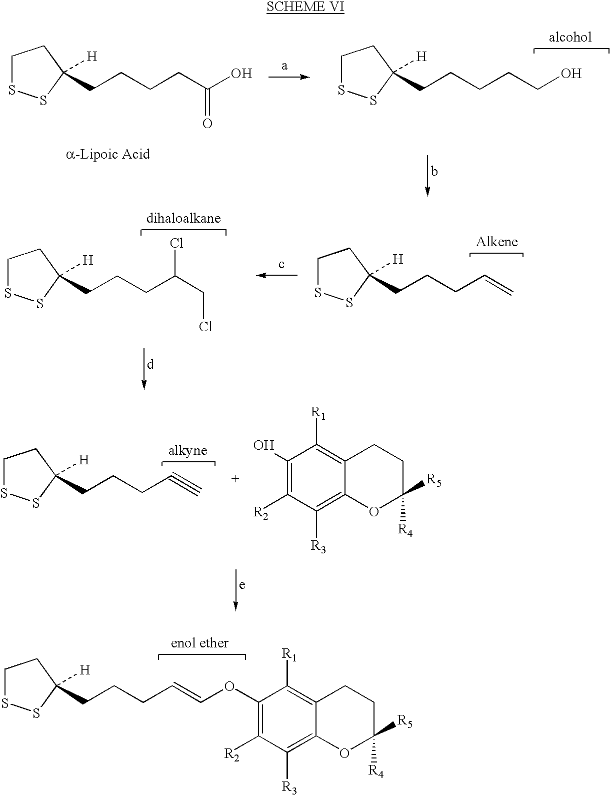

Step “a” can be carried out with LiAlH₄, for example, as described in House, which is hereby incorporated by reference, followed by treatment with H₂O₂, for example, as described in Capozi, which is hereby incorporated by reference. Step “b” can be carried out using, e.g., H₂So₄, for example, as described in March, Advanced Organic Chemistry, 3rd ed., New York: John Wiley & Sons, p. 901 (1985), which is hereby incorporated by reference. Step “c” can be carried our with Cl₂, for example, following the procedures set forth in de la Mare, Electrophilic Halogenation, London: Cambridge University Press (1976), which is hereby incorporated by reference. Step “d” can be carried out with NaNH₂, for example, as described in March, Advanced Organic Chemistry, 3rd ed., New York: John Wiley & Sons, p. 915 (1985), which is hereby incorporated by reference. Step “e”, reaction of the alkyne with the phenol functionality of the modified benzopyran (e.g., α-tocopherol), can be carried out, for example, using the procedures described in Shostakovskii et al., Russ. Chem. Rev., 37:907-919 (1968), which is hereby incorporated by reference. In the method illustrated in Scheme VI, the carboxylic acid functionality of the a-lipoic acid is reduced, e.g., with LiAlH₄. Under certain conditions, the reducing conditions employed may reduce the disulfide to the sulfhydryl derivative, in which case oxidation back to the disulfide can be accomplished with, for example, hydrogen peroxide (Step “a”). The resulting alcohol can then be reacted with acid to produce the alkene (Step “b”), and electrophilic halogenation of the alkene can be used to generate the dihaloalkane (Step “c”). Elimination of the dihaloalkane, using, for example, NaNH₂ or other suitable strong base, produced the alkyne (Step “d”). The resulting alkyne can then be treated with a phenol (e.g., α-tocopherol) to produce the enol ether analog (Step “e”).

It will be appreciated that Schemes I-VI are general and can be readily extended to other benzopyrans (e.g., benzopyrans bearing substituents on the carbon directly across from the benzopyran's ring oxygen atom) and to other disulfides (e.g., disulfides which are part of 6-, 7-, or 8-membered rings, disulfide-containing rings whose ring carbon atoms are substituted, and/or disulfide-containing rings bearing side chains of varying length and/or side chains which are substituted with one or more substituents).

The present invention also relates to compounds which include a water-soluble antioxidant that is covalently bonded, directly or indirectly, to a lipid-soluble antioxidant.

As used in this embodiment, an antioxidant is to be deemed to be “lipid-soluble” if (i) its lipid solubility is at least about 50% that of α-tocopherol (e.g., as in the case where the lipid-soluble antioxidant has a lipid solubility of at least about 60% that of α-tocopherol, at least about 70% that of α-tocopherol, at least about 80% that of α-tocopherol, at least about 90% that of α-tocopherol, at least about 100% that of α-tocopherol, and/or greater than that of α-tocopherol) or (ii) its water-octanol partition coefficient, P (where P=[antioxidant]_{octanol}/[antioxidant]_{water}), is greater than about 3.5, such as greater than about 4, greater than about 4.5, greater than about 5, greater than about 5.5, greater than about 6, greater than about 6.5, greater than about 7, greater than about 7.5, and/or greater than about 8. Illustrative lipid-soluble antioxidants include those described above. For example, the lipid-soluble antioxidant can be a tocopherol ring system which is substituted with at least one lipophilic moiety and which is otherwise substituted or unsubstituted.

As used in this embodiment, an antioxidant is to be deemed to be “water-soluble” if its water solubility is at least about 50% that of lipoic acid (e.g., as in the case where the water-soluble antioxidant has a solubility in water of at least about 60% that of lipoic acid, at least about 70% that of lipoic acid, at least about 80% that of lipoic acid, at least about 90% that of lipoic acid, at least about 100% that of lipoic acid, and/or greater than that of lipoic acid). Illustrative water-soluble antioxidants include cyclic and acyclic disulfides as well as reduced sulfhydryl derivatives of such disulfides, examples of which have been provided above.

The terms and phrases “compound”, “antioxidant”, “covalently bonded”, and the like, as used in this embodiment, have the meanings ascribed to them above. Illustratively, the water-soluble antioxidant can be covalently bonded to the lipid-soluble antioxidant via a bridging moiety which contains an ester, an amide, a carbamate, a carbonate, an imine, a urea, or an enol ether functional group.

The compounds and reduced sulfhydryl derivatives of the present invention can be used to inhibit oxidative and/or free radical damage in cells by contacting the cells with an effective amount of the compound or of the reduced sulfhydryl derivative. The method can be carried out in vitro, for example to preserve tissue samples. When practiced in vitro, the cells (e.g., the cells of a tissue sample) can be placed in a test tube, beaker, petri dish, or other suitable container, and contacting can be carried out simply by adding the compound (or the reduced sulfhydryl derivative) of the present invention to the cells (e.g., by dissolving or suspending the compound (or the reduced sulfhydryl derivative) in a suitable solvent and mixing the resulting solution or suspension with the cells). Alternatively, the method can be carried out in vivo, for example, in a subject, such as a mouse, rat, cat, dog, pig, goat, sheep, horse, human, or other mammal. This can be carried out, illustratively, by directly injecting the compound or the reduced sulfhydryl derivative (e.g., in a suitable vehicle) into a tissue (of the subject) which contains the cells where oxidative and/or free radical damage inhibition is desired.

The compounds and reduced sulfhydryl derivatives of the present invention can be used to inhibit oxidative and/or free radical damage in a subject's cells by administering (e.g., orally, subcutaneously, intraperitoneally, intravenously, intramuscularly, etc.) a compound or a reduced sulfhydryl derivative of the present invention to the subject under conditions effective to inhibit oxidative and/or free radical damage in a subject's cells.

As used herein, “inhibit” is meant to include total inhibition of (i.e., 100% reduction in) oxidative and/or free radical damage as well as partial inhibition of oxidative and/or free radical damage (e.g., a reduction of between about 10% and 100%, such as a reduction of between about 20% and 100%, a reduction of between about 30% and 100%, a reduction of between about 40% and 100%, a reduction of between about 50% and 100%, a reduction of between about 60% and 100%, a reduction of between about 70% and 100%, a reduction of between about 80% and 100%, a reduction of between about 90% and 100% in oxidative and/or free radical damage), as measured, for example, by using standard assays for antioxidant activity, such as the inhibition of ferrous ion-stimulated formation of maliondialdehyde in microsomes or liposomes.

As used herein, “oxidative and/or free radical damage” is meant to include damage which is the result of hypoxia, damage which is the result of ischemia, damage which is the result of reoxygenation injury, damage which is the result of calcium released from the sarcoplasmic reticulum, damage which is the result of lipid peroxidases, damage which is the result of a calcium-activated-protease (e.g., calpain), damage which is the result of a calcium-activated lipase (e.g., phospholipase A₂), damage which is the result of reactive nitrogen species, damage which is the result of reactive oxygen species, and damage which is the result of combinations thereof. As used in this context, the phrase “is the result of” is meant to include direct results as well as indirect results.

Suitable subjects include, for example, mice, rats, humans, and other mammals, such as mice, rats, humans, and other mammals who are suffering from and/or are likely to be suffering from and/or are susceptible to and/or are likely to be susceptible to hypoxia or ischemia. Illustratively, suitable subjects can include mice, rats, humans, and other mammals who are suffering from and/or are likely to be suffering from and/or are susceptible to and/or are likely to be susceptible to stroke, heart attack, heart disease, coronary artery disease, vascular disease, peripheral vascular disease, cardiovascular disease, hypertension, atherosclerosis, diabetes, diabetic neuropathy, bladder dysfunction, brain disorders, neurodegenerative diseases, Alzheimer's disease, dementia, inflammation, autoimmune disease, arthritis, diseases or disorders involving oxidative or free radical attack on mitochondria, and/or diseases or disorders involving oxidative or free radical attack on neural membranes. As a further illustration, suitable subjects can include mice, rats, humans, and other mammals who are suffering from and/or are likely to be suffering from and/or are susceptible to and/or are likely to be susceptible to the diseases, syndromes, and other conditions set forth in Halliwell et al., Free Radicals in Biology and Medicine, 2nd ed., Oxford: Clarendon Press, pp. 416-449 (1989) (“Halliwell”), which is hereby incorporated by reference. In the context of bladder dysfunction, suitable subjects include those who (i) exhibit progressive denervation, e.g., as evidenced by decreased choline acetyl transferase activity (which can be measured, for example, using the methods described in Roelofs et al., “Contractility and Phenotype Transitions in Serosal Thickening of Obstructed Rabbit Bladder,” J. Applied Physiol., 78:1432-1441 (1995) and Levin et al., “Effect of Partial Outlet Obstruction on Choline Acetyltransferase Activity in the Rat and Rabbit,” Neurourol. Urodyn., 12:255-262 (1993), which are hereby incorporated by reference); (ii) exhibit denervation as evidenced by specific electron microscopic analyses such as those described in Levin et al., “Obstructive Response of Human Bladder to BPH vs. Rabbit Bladder Response to Partial Outlet Obstruction: A Direct Comparison,” Neurourol. Urodyn., 19:609-629 (2000) (“Levin II”), Gosling et al., “Correlation Between the Structure and Function of the Rabbit Urinary Bladder Following Partial Outlet Obstruction,” J. Urol., 163:1349-1356 (2000) (“Gosling I”), and Gosling et al., “Modification of Bladder Structure in Response to Outflow Obstruction and Ageing,” Eur. Urol., 32(Suppl. 1):9-14 (1997) (“Gosling II”), which are hereby incorporated by reference; (iii) exhibit selective dysfunction of the sarcoplasmic reticulum, as evidenced, for example, by decreased thapsigargin sensitive calcium ATPase activity (“SERCA”) (which can be measured, for example, using the methods described in Haugaard et al., “Properties of Ca²⁺—Mg²⁺ATP-ase in Rabbit Bladder Muscle and Mucosa: Effect of Urinary Outlet Obstruction,” Neurourol. Urodyn., 15:555-561 (1996) and Zderic et al., “The Decompensated Detrusor II: Evidence for Loss of Sarcoplasmic Reticulum Function Following Bladder Outlet Obstruction in the Rabbit,” J. Urol., 156:587-592 (1996), which are hereby incorporated by reference); (iv) exhibit selective mitochondrial dysfunction, as evidenced, for example, by decreased citrate synthase activity (which can be measured, for example, using the methods described in Haugaard et al., “Effect of Partial Obstruction of the Rabbit Urinary Bladder on Malate Dehydrogenase and Citrate Synthase Activity,” J. Urol., 147:1391-1393 (1992); Hypolite et al., “Effect of Partial Outlet Obstruction on ¹⁴C-adenine Incorporation in the Rabbit Urinary Bladder,” Neurourol. Urodyn., 16:201-208 (1997); and Zhao et al., “Partial Outlet Obstruction of the Rabbit Bladder Results in Changes in the Mitochondrial Genetic System,” Mol. Cell Biochem., 141:47-55 (1994) (Zhao″), which are hereby incorporated by reference); and/or (v) exhibit mitochondrial damage as evidenced by electron microscopic analyses and/or molecular studies such as those described in Wang et al., “Loss of Mitochondrial DNA in Rabbit Bladder Smooth Muscle Following Partial Outlet Obstruction Results from Lack of Organellar DNA Replication,” Mol. Urol., 5:99-104 (2001), Nevel-McGarvey et al., “Mitochondrial and Mitochondrial-related Nuclear Genetic Function in Rabbit Urinary Bladder Following Reversal of Outlet Obstruction,” Mol. Cell Biochem., 197:161-172 (1999), Nevel-McGarvey et al., “Transcription of Mitochondrial and Mitochondrial-related Nuclear Genes in Rabbit Bladder Following Partial Outlet Obstruction,” Mol. Cell Biochem., 173:95-102 (1997), Levin II, Gosling I, Gosling II, and Zhao, which are hereby incorporated by reference.

As indicated above, the method of the present invention for inhibiting oxidative and/or free radical damage in a subject's cells includes administering a compound or a reduced sulfhydryl derivative of the present invention to the subject under conditions effective to inhibit oxidative and/or free radical damage in a subject's cells. Suitable routes of administration include, for example, oral, subcutaneous, intraperitoneal, intramuscular, etc.

The present invention, in another aspect thereof, relates to a method of inhibiting oxidative and/or free radical damage in a subject's nerve membranes, sarcoplasmic reticula, mitochondrial membranes, and/or muscle plasma membranes. The method includes administering a compound or a reduced sulfhydryl derivative of the present invention to the subject under conditions effective to inhibit oxidative and/or free radical damage in the subject's nerve membranes, sarcoplasmic reticula, mitochondrial membranes, and/or muscle plasma membranes.

The present invention, in another aspect thereof, relates to a method of treating or preventing, in a subject, a disease, syndrome, disorder, or other condition involving ischemia, hypoxia, and/or reoxygenation injury. Examples of such conditions include stroke, heart attack, heart disease, coronary artery disease, vascular disease, peripheral vascular disease, cardiovascular disease, hypertyension, atherosclerosis, diabetes, diabetic neuropathy, bladder dysfunction, obstructive bladder disease, ischemic bladder disease, brain disorders, neurodegenerative diseases, Alzheimer's disease, dementia, inflammation, autoimmune disease, arthritis, diseases or disorders involving oxidative or free radical attack on mitochondria, diseases or disorders involving oxidative or free radical attack on neural membranes, and/or the diseases, syndromes, disorders, or other conditions set forth in Halliwell which is hereby incorporated by reference. The method includes administering an effective amount of a compound or a reduced sulfhydryl derivative of the present invention to the subject.

Illustratively, the treatment/prevention method of the present invention can be used to treat or prevent obstructive and ischemic bladder diseases in a subject by administering an effective amount of a compound or a reduced sulfhydryl derivative of the present invention to the subject. Typically, effective amounts, as used in the context of treating obstructive and ischemic bladder diseases, include those which (i) reverse the effects of mild partial outlet obstruction and ischemia; (ii) increase the compliance of obstructed bladders; and/or (iii) improve the contractile responses of obstructed bladders. Typically, effective amounts, as used in the context of preventing obstructive and ischemic bladder diseases, include those which prevent or reverse the progression from compensated bladder function to decompensated bladder function. Suitable methods for assessing the effectiveness of treatment and prevention of obstructive and ischemic bladder diseases in terms of reversing the effects of mild partial outlet obstruction and ischemia; increasing the compliance of obstructed bladders; improving the contractile responses of obstructed bladders; and/or preventing or reversing the progression from compensated bladder function to decompensated bladder function can be found, for example, in Kato, which is hereby incorporated by reference, and/or in Levin I, which is hereby incorporated by reference.

It should be noted that the compounds of the present invention (or their reduced sulfhydryl derivatives) and those compounds (or reduced sulfhydryl derivatives) used in the methods of the present invention, when administered to the subject, can itself be active (e.g., as an anti-oxidant), or, under certain conditions (e.g., in the case where the compound contains a bridging group having a linking group susceptible to metabolic cleavage, such as by hydrolysis or reduction), the administered compound can be cleaved in vivo to produce two actives (e.g., one which derives from the cyclic or acyclic disulfide and the other which derives from the lipid-soluble antioxidant).

The compounds of the present invention (or their reduced sulfhydryl derivatives) and those compounds (or reduced sulfhydryl derivatives) used in the methods of the present invention can be administered alone or in combination with suitable pharmaceutical carriers or diluents. The diluent or carrier ingredients should be selected so that they do not diminish the desired effects of the compounds of the present invention (or their reduced sulfhydryl derivatives). The compounds or their reduced sulfhydryl derivatives can be made up in any suitable form appropriate for the desired use. Where they are to be used in vivo, they can be formulated for any conventional route of administration, such as oral, parenteral, or topical administration. Examples of parenteral administration are intraventricular, intracerebral, intramuscular, intranasal, intravenous, intraperitoneal, rectal, and subcutaneous administration. While enteral (e.g., oral) administration is generally preferred, the choice of administration route can depend on the location of the oxidative and/or free radical damage to be inhibited. For example, in the case where inhibition of oxidative and/or free radical damage in lung tissue is desired, intranasal administration can be employed. Suitable dosage forms for oral use include tablets, dispersible powders, granules, capsules, suspensions, syrups, and elixirs. Inert diluents and carriers for tablets include, for example, calcium carbonate, sodium carbonate, lactose, and talc. Tablets may also contain granulating and disintegrating agents, such as starch and alginic acid; binding agents, such as starch, gelatin, and acacia; and lubricating agents, such as magnesium stearate, stearic acid, and talc. Tablets may be uncoated or may be coated by known techniques to delay disintegration and absorption. Inert diluents and carriers which may be used in capsules include, for example, calcium carbonate, calcium phosphate, and kaolin. Suspensions, syrups, and elixirs may contain conventional excipients, such as methyl cellulose, tragacanth, sodium alginate; wetting agents, such as lecithin and polyoxyethylene stearate; and preservatives, such as ethyl-p-hydroxybenzoate.

Dosage forms suitable for parenteral administration include solutions, suspensions, dispersions, emulsions, and the like. They may also be manufactured in the form of sterile solid compositions which can be dissolved or suspended in sterile injectable medium immediately before use. They may contain suspending or dispersing agents or other excipients known in the art, such as the ones further discussed below.

For oral administration either solid or fluid unit dosage forms can be prepared. For preparing solid compositions, such as tablets, a suitable compound or reduced sulfhydryl derivative, as disclosed above, is mixed with conventional ingredients, such as talc, magnesium stearate, dicalcium phosphate, magnesium aluminum silicate, calcium sulfate, starch, lactose, acacia methylcellulose, and functionally similar materials as pharmaceutical diluents or carriers. Capsules are prepared by mixing the disclosed compound or reduced sulfhydryl derivative with an inert pharmaceutical diluent and filling the fixture into a hard gelatin capsule of appropriate size. Soft gelatin capsules are prepared by machine encapsulation of a slurry of the compound with an acceptable vegetable oil, light liquid petrolatum, or other inert oil. Fluid unit dosage forms for oral administration such as syrups, elixirs, and suspensions can be prepared by dissolving the compound in suitable solvent together with sugar, aromatic flavoring agents, and preservatives to form a syrup. An elixir is prepared by using a hydro-alcoholic (ethanol) vehicle with suitable sweeteners, such as sugar and saccharin, together with an aromatic flavoring agent. Suspensions can be prepared with a syrup vehicle with the aid of a suspending agent, such as acacia, tragacanth, methylcellulose, and the like.

In addition to the above, generally non-active ingredients, the dosage forms can also (i.e., in addition to a compound or reduced sulfhydryl derivative of the present invention) contain other active pharmaceutical agents, for example, pharmaceutical agents which are commonly used to treat or alleviate the symptoms of the disease or disorder from which the subject suffers. For example, where the subject suffers from obstructive bladder disease, the dosage forms can further include materials which have been shown to be effective in the treatment of symptoms of obstructive bladder disease, such as Tadenan (an extract from the bark of the African plum tree, Pygeum africanum).

For parenteral administration, fluid unit dosage forms are prepared utilizing the aforementioned compounds (or their reduced sulfhydryl derivatives) and a sterile vehicle. The compound or reduced sulfhydryl derivative, depending on the vehicle and concentration used, can be either suspended or dissolved in the vehicle. In preparing solutions, the compound or reduced sulfhydryl derivative can be dissolved in a suitable solvent for injection and filter sterilized before filling into a suitable vial or ampule and sealing. Advantageously, adjuvants, such as a local anesthetic, preservative, and buffering agents, can be dissolved in the vehicle. To enhance the stability, the composition can be frozen after filling into the vial, and the solvent removed under vacuum. The resulting powder is then sealed in the vial, and an accompanying vial of solvent for injection is supplied to reconstitute the liquid prior to use. Parenteral suspensions are prepared in substantially the same manner, except that the compound or reduced sulfhydryl derivative is suspended in the vehicle instead of being dissolved, and sterilization cannot be accomplished by filtration. The compound or reduced sulfhydryl derivative can be sterilized by exposure to ethylene oxide before suspending in the sterile vehicle.

Irrespective of the route of administration, suitable daily dosages can be ascertained by standard methods, such as by establishing dose-response curves in laboratory animal models or clinical trials.

Although the above discussion illustrates the methods and pharmaceutical compositions of the present invention by discussing the administration of one compound or of one reduced sulfhydryl derivative, it will be appreciated that the methods and pharmaceutical compositions of the present invention can be practiced with a plurality of compounds according to the present invention, with a plurality of reduced sulfhydryl derivatives according to the present invention, or with any combination of compounds and reduced sulfhydryl derivatives according to the present invention.

The present invention is further illustrated by the following examples.

### EXAMPLES

### Example 1

### Preparation of Compound MH-1

Compound MH-1 was synthesized according to the procedure set forth below.

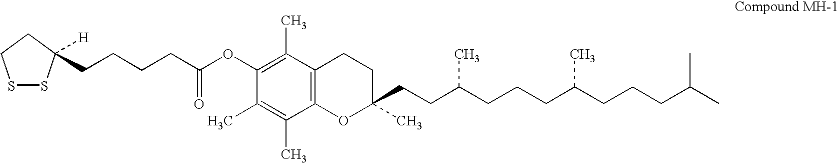

α-Tocopherol (4.3 g, 10 mmol) in 50 ml of CH₂Cl₂ was added to lipoic acid (2.06 g, 10 mmol) in 50 ml of CH₂Cl₂. An excess of dicyclohexylcarbodiimide (“DCC”) (8.25 g, 40 mmol) in 20 ml of CH₂Cl₂ was added to the reaction mixture. 4-Dimethylaminopyridine (“DMAP”) (1 mg) was added to the reaction mixture. The reaction mixture was allowed to stir at room temperature for 24 hr. Reaction progress was monitored by thin layer chromatography (Silica gel, EtOAc:Hexane, 50:50, UV, I₂).

The crude product was shown to be contaminated with unreacted α-tocopherol, unreacted lipoic acid, unreacted DCC, and dicyclohexylurea. Purification of the crude product was carried out by flash column chromatography (Silica gel, EtOAc:Hexane, 50:50) to yield a pale yellow solid (2.6 g, 42%).

### Example 2

### Antioxidant Activity of Compound MH-1

The in vitro antioxidant activity of Compound MH-1 was evaluated by measuring its inhibitory effect on the ferrous ion-stimulated formation of maliondialdehyde (“MDA”), an end product of lipid peroxidation, in rat liver microsomes. The antioxidant activity of Compound MH-1 was compared with the in vitro antioxidant activity of α-tocopherol, a known antioxidant. A modified procedure originally described in Bernheim et al., “The Reaction Between Thiobarbituric Acid and the Oxidation Products of Certain Lipids,” J. Biol. Chem., 174:257-264 (1948) and Wills, “Lipid Peroxide Formation in Microsomes,” Biochem. J., 113:325-332 (1969), which are hereby incorporated by reference, was used to carry out the assay.

Freshly harvested rat livers (100 mg/ml) were homogenized in Tris-KCl buffer (0.05M, pH 7.4). The microsomal fraction was isolated by differential centrifugation (40,000 rpm; 106,000 g) and resuspended in Tris-KCl (100 mg/ml, initial concentration). The microsomal suspension was incubated with or without the test compound (Compound MH-1 in DMSO, 0.0-0.5mM or α-tocopherol in dimethylsulfoxide, 0.0-0.6 mM) at 37° C. for 3 minutes. Ferrous sulfate (50 ml, final concentration 1 mM) was added to the microsomal suspension to initiate lipid peroxidation. The mixture was incubated at 37° C. for 1 hour. The reaction was terminated by addition of 40% trifluoroacetic acid. The mixture was centrifuged, and aliquots of the supernatant (100 ml) were combined with thiobarbituric acid (“TBA”) (0.75 ml, 1% in water). The reaction was incubated at 90° C. for 30 minutes, cooled on ice, and extracted with n-butanol. Maliondialdeyde-TBA adduct concentrations in the butanol extract were measured by fluorescence spectroscopy (emission 553 nm, excitation 532 nm). Tetraethoxypropane reacted with TBA at various concentrations was used to generate a standard curve.

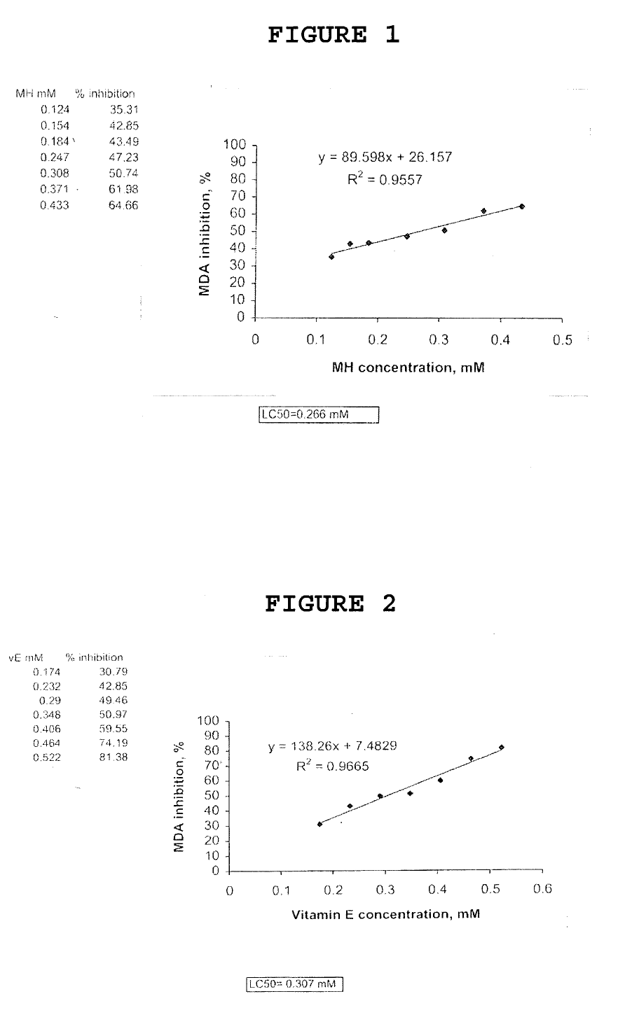

The results demonstrate that Compound MH-1 inhibited production of MDA (as measured by the MDA-TBA adduct) with an IC₅₀=0.266mM (FIG. 1). Inhibition of MDA production by α-tocopherol was also observed with an IC₅₀=0.307 mM. (FIG. 2).

Although the invention has been described in detail for the purpose of illustration, it is understood that such detail is solely for that purpose, and variations can be made therein by those skilled in the art without departing from the spirit and scope of the invention which is defined by the following claims.

What is claimed is:

1. A compound comprising a cyclic or acyclic disulfide covalently bonded, directly or indirectly, to a lipid-soluble antioxidant; or a reduced sulfhydryl derivative of said compound.

2. A compound according to claim 1, in which the cyclic or acyclic disulfide is covalently bonded to the lipid-soluble antioxidant via a bridging moiety which contains an ester, an amide, a carbamate, a carbonate, an imine, a urea, or an enol ether functional group; or a reduced sulfhydryl derivative of said compound.

3. A compound according to claim 1, wherein the cyclic or acyclic disulfide is an acyclic disulfide.

4. A compound according to claim 1, wherein the cyclic or acyclic disulfide is a cyclic disulfide

5. A compound according to claim 1 having the formula: 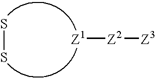
wherein Z¹ represents a substituted or unsubstituted C2-C6 alkylene moiety, Z² represents a bridging moiety, and Z³ represents the lipid-soluble antioxidant; or a reduced sulfhydryl derivative of said compound.

6. A compound according to claim 5, wherein Z¹ represents a substituted or unsubstituted C3-C5 alkylene moiety; or a reduced sulthydryl derivative of said compound.

7. A compound according to claim 5, wherein Z'has the formula: 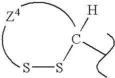
wherein Z⁴ represents a substituted or unsubstituted C2-C5 alkylene moiety; or a reduced sulfhydryl derivative of said compound.

8. A compound according to claim 7, wherein Z⁴ represents an unsubstituted C2-C5 alkylene moiety; or a reduced sulfhydryl derivative of said compound.

9. A compound according to claim 5, wherein Z¹ has the formula: 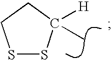
or a reduced sulfhydryl derivative of said compound.

10. A compound according to claim 5, wherein Z³ represents a tocopherol ring system which is substituted with at least one lipophilic moiety and which is otherwise substituted or unsubstituted; or a reduced sulfhydryl derivative of said compound.

11. A compound according to claim 5, wherein Z³ represents an α-tocopherol moiety, β-tocopherol moiety, a γ-tocopherol moiety, a δ-tocopherol moiety, an ζ₁-tocopherol moiety, an ζ₂-tocopherol moiety, an η-tocopherol moiety, or a tocol moiety and wherein said α-tocopherol moiety, β-tocopherol moiety, γ-tocopherol moiety, δ-tocopherol moiety, ζ₁-tocopherol moiety, ζ₂-tocopherol moiety, δ-tocopherol moiety, or tocol moiety is covalently bonded to Z² via its hydroxyl carbon; or a reduced sulfhydryl derivative of said compound.

12. A compound according to claim 5, wherein Z³ has the formula: 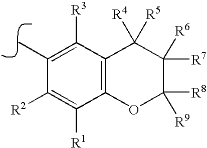
wherein R¹-R⁹ are independently selected from the group consisting of hydrogen, a substituted or unsubstituted alkyl, a substituted or unsubstituted 4-8 membered homocyclic ring, a substituted or unsubstituted 4-8 membered heterocyclic ring, a hydroxy group, a substituted or unsubstituted alkoxy group, a substituted or unsubstituted amine group, a halogen, a carboxylic acid group, a carboxylic acid ester group, and a carboxylic acid amide group, provided that at least one of R¹-R⁹ is a lipophilic moiety; or a reduced sulfhydryl derivative of said compound.

13. A compound according to claim 12, wherein R¹-R⁹ are independently selected from the group consisting of hydrogen and substituted or unsubstituted alkyls, provided that at least one of R¹-R⁹ is a lipophilic moiety; or a reduced sulthydryl derivative of said compound.

14. A compound according to claim 12, wherein R¹-R³ and R⁹ are independently selected from the group consisting of substituted alkyls and unsubstituted alkyls, wherein one of R⁴-R⁸ is a substituted or unsubstituted lipophilic alkyl or aryl, and wherein the remaining of R⁴-R⁸ are hydrogen; or a reduced sulfhydryl derivative of said compound.

15. A compound according to claim 12, wherein each of R¹-R³ and R⁹ is a methyl group, wherein one of R⁴-R⁸ is a substituted or unsubstituted lipophilic alkyl or aryl, and wherein the remaining of R⁴-R⁸ are hydrogen, or a reduced sulfhydryl derivative of said compound.

16. A compound according to claim 12, wherein each of R¹-R³ and R⁹ is a methyl group, wherein each of R⁴-R⁷ is hydrogen, and wherein R⁸ is a substituted or unsubstituted lipophilic alkyl; or a reduced sulfhydryl derivative of said compound.

17. A compound according to claim 12, wherein each of R¹-R³ and R⁹ is a methyl group, wherein each of R⁴-R⁷ is hydrogen, and wherein R⁸ is an unsubstituted lipophilic alkyl, or a reduced sulfhydryl derivative of said compound.

18. A compound according to claim 12, wherein each of R¹-R³ and R⁹ is a methyl group, wherein each of R⁴-R⁷ is hydrogen, and wherein R⁸ is a 3,7,11-trimethyldodecyl group; or a reduced sulfhydryl derivative of said compound.

19. A compound according to claim 1, in which the cyclic or acyclic disulfide is covalently bonded to the lipid-soluble antioxidant via a bridging moiety having the formula:
—Z⁵—Z⁶—
wherein Z⁵ represents a substituted or unsubstituted C1-C8 alkylene moiety and Z⁶ represents an ester, an amide, a carbamate, a carbonate, an imine, a urea, or an enol ether functional group; or a reduced sulfhydryl derivative of said compound.

20. A compound according to claim 1, in which the cyclic or acyclic disulfide is covalently bonded to the lipid-soluble antioxidant via a bridging moiety having the formula:
—Z⁵—Z⁶—
wherein Z⁵ represents a substituted or unsubstituted C3-C5 alkylene moiety and Z⁶ represents an ester functional group; or a reduced sulfhydryl derivative of said compound.

21. A compound according to claim 1 having the formula: 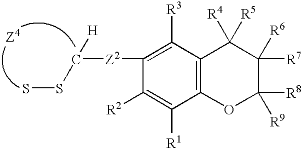
wherein Z⁴ represents a substituted or unsubstituted C2-C5 alkylene moiety, wherein Z² bridging moiety, and wherein R¹-R⁹ are independently selected from the group consisting of hydrogen, a substituted or unsubstituted alkyl, a substituted or unsubstituted 4-8 membered homocyclic ring, a substituted or unsubstituted 4-8 membered heterocyclic ring, a hydroxy group, a substituted or unsubstituted alkoxy group, a substituted or unsubstituted amine group, a halogen, a carboxylic acid group, a carboxylic acid ester group, and a carboxylic acid amide group, provided that at least one of R¹-R⁹ is a lipophilic moiety; or a reduced sulfhydryl derivative of said compound.

22. A compound according to claim 21, wherein R¹-R⁹are independently selected from the group consisting of hydrogen and substituted or unsubstituted alkyls, provided that at least one of R¹-R⁹ is a lipophilic moiety; or a reduced sulfhydryl derivative of said compound.

23. A compound according to claim 21, wherein R¹-R³ and R⁹ are independently selected from the group consisting of hydrogen, substituted alkyls, and unsubstituted alkyls, wherein one of R⁴-R⁸ is a substituted or unsubstituted lipophilic alkyl or aryl, and wherein the remaining of R⁴-R⁸ are hydrogen; or a reduced sulfhydryl derivative of said compound.

24. A compound according to claim 21, wherein R¹-R³ and R⁹ are independently selected from the group consisting of hydrogen and a methyl group, wherein one of R⁴-R⁸ is a substituted or unsubstituted lipophilic alkyl or aryl, and wherein the remaining of R⁴-R⁸ are hydrogen; or a reduced sulfhydryl derivative of said compound.

25. A compound according to claim 21, wherein each of R¹-R³ and R⁹is a methyl group, wherein each of R⁴-R⁷ is hydrogen, and wherein R⁸ is a substituted or unsubstituted lipophilic alkyl; or a reduced sulfhydryl derivative of said compound.

26. A compound according to claim 21, wherein each of R¹-R³ and R⁹is a methyl group, wherein each of R⁴-R⁷ is hydrogen, and wherein R⁸ is an unsubstituted lipophilic alkyl; or a reduced sulfhydryl derivative of said compound.

27. A compound according to claim 21, wherein each of R¹-R³ and R⁹ is a methyl group, wherein each of R⁴-R⁷ is hydrogen, and wherein R⁸ is a 3,7,11-trimethyldodecyl group; or a reduced sulfhydryl derivative of said compound.

28. A compound according to claim 21, in which Z² has the formula:
—Z⁵—Z⁶—
wherein Z⁵ represents a substituted or unsubstituted C1-C8 alkylene moiety and Z⁶ represents an ester, an amide, a carbamate, a carbonate, an imine, a urea, or enol ether functional group; or a reduced sulfhydryl derivative of said compound.

29. A compound according to claim 21, in which Z² had the formula:
—Z⁵—Z⁶—
wherein Z⁵ represents a substituted or unsubstituted C3-C5 alkylene moiety and Z⁶ represents an ester functional group; or a reduced sulfhydryl derivative of said compound.

30. A compound according to claim 21, wherein Z⁴ is a —CH₂—CH₂— group, wherein each of R¹-R³ and R⁹ is a methyl group, wherein each of R⁴-R⁷ is hydrogen, wherein R⁸ is a 3,7,11-trimethyldodecyl group, wherein Z² has the formula —(CH₂)ₙ—Z⁶—, wherein n is an integer from 1 to 8, and wherein Z⁶ represents an ester, an amide, a carbamate, a carbonate, an imine, a urea, or enol ether linkage; or a reduced sulfhydryl derivative of said compound.

31. A compound according to claim 21, wherein Z⁴ is a —CH₂—CH₂— group, wherein each of R¹-R³ and R⁹ is a methyl group, wherein each of R⁴-R⁷ is hydrogen, wherein R⁸ is a 3,7,11-trimethyldodecyl group, wherein Z² has the formula —(CH₂)ₙ—C(O)O—, and wherein n is an integer from 3 to 5; or a reduced sulfhydryl derivative of said compound.

32. A compound according to claim 21 having a formula selected from the following group of formulae: 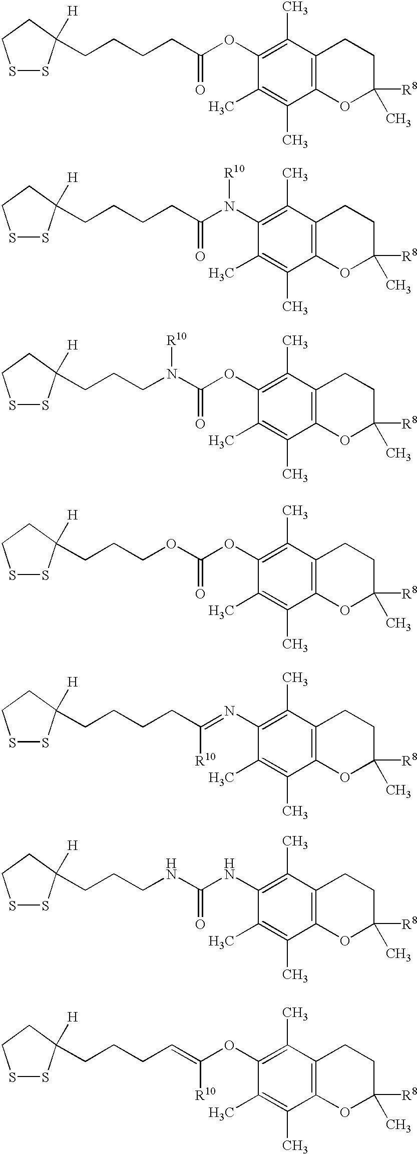
wherein R⁸ is an unsubstituted lipophilic alkyl and wherein R¹⁰ is selected from the group consisting of hydrogen, substituted or unsubstituted alkyl, and substituted or unsubstituted aryl; or a reduced sulfhydryl derivative of said compound.

33. A compound according to claim 32, wherein R⁸ is a 3,7,11-trimethyldodecyl group; or a reduced sulfhydryl derivative of said compound.

34. A compound according to claim 21 having the formula: 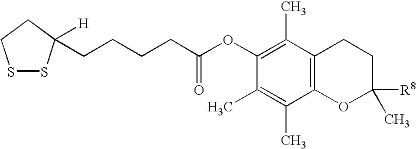
wherein R⁸ is an unsubstituted lipophilic alkyl; or a reduced sulfhydryl derivative of said compound.

35. A compound according to claim 34, wherein R⁸ is a 3,7,11-trimethyldodecyl group; or a reduced sulfhydryl derivative of said compound.

36. A compound according to claim 21 having a formula selected from the following group of formulae: 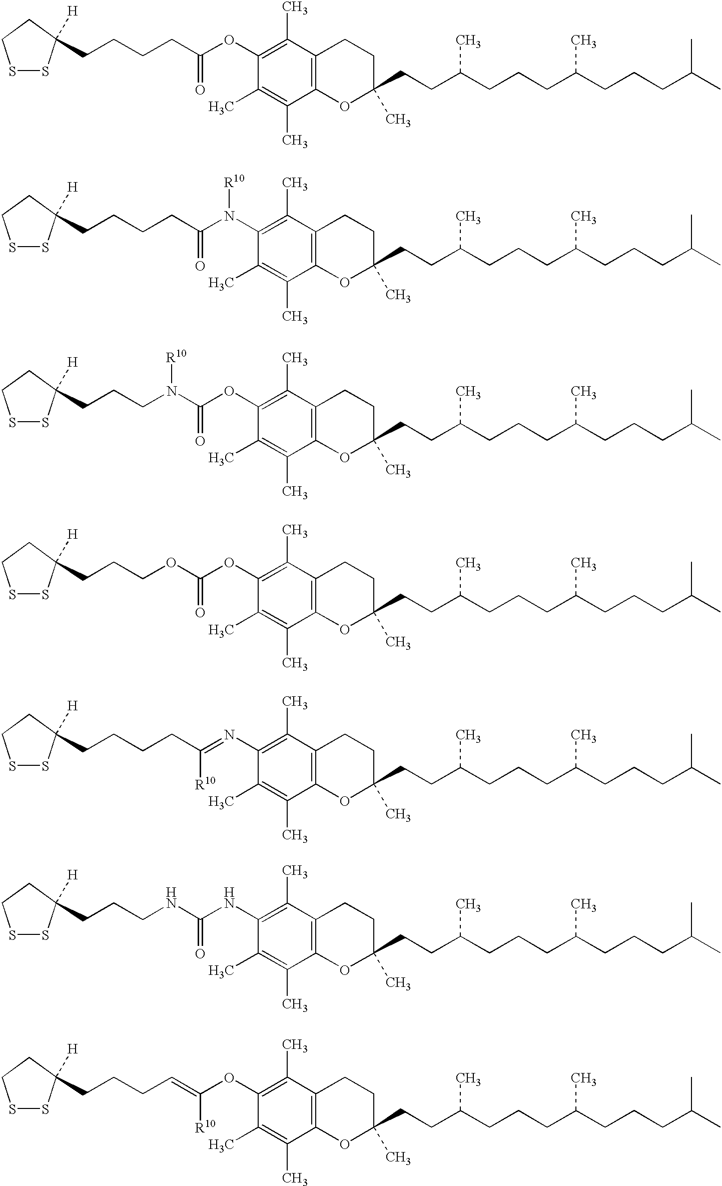
wherein R¹⁰ is selected from the group consisting of hydrogen, substituted or unsubstituted alkyl, and substituted or unsubstituted aryl; or a reduced sulfhydryl derivative of said compound.

37. A compound according to claim 21 having the formula: 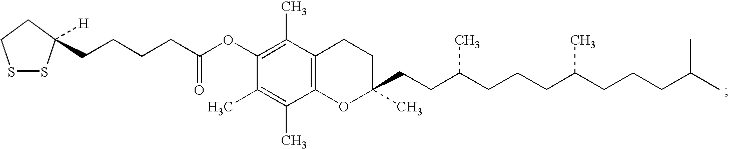
or a reduced sulfhydryl derivative of said compound.

38. A compound comprising a water-soluble antioxidant that is covalently bonded, directly or indirectly, to a lipid-soluble antioxidant.

39. A compound according to claim 38, wherein the water-soluble antioxidant is covalently bonded to the lipid-soluble antioxidant via a bridging moiety which contains an ester, an amide, a carbamate, a carbonate, an imine, a urea, or an enol ether functional group.

40. A compound according to claim 38, wherein the lipid-soluble antioxidant is a tocopherol ring system which is substituted with at least one lipophilic moiety and which is otherwise substituted or unsubstituted.

41. A pharmaceutical composition comprising:
a compound according to claim 1 or a reduced sulfhydryl derivative of said compound; and
a pharmaceutically acceptable carrier.

42. A pharmaceutical composition comprising:
a compound according to claim 5 or a reduced sulfhydryl derivative of said compound; and
a pharmaceutically acceptable carrier.

43. A pharmaceutical composition comprising:
a compound according to claim 7 or a reduced sulfhydryl derivative of said compound; and
a pharmaceutically acceptable carrier.

44. A pharmaceutical composition comprising:
a compound according to claim 12 or a reduced sulfhydryl derivative of said compound; and
a pharmaceutically acceptable carrier.

45. A pharmaceutical composition comprising:
a compound according to claim 21 or a reduced sulfhydryl derivative of said compound; and
a pharmaceutically acceptable carrier.

46. A pharmaceutical composition comprising:
a compound according to claim 32 or a reduced sulfhydryl derivative of said compound; and
a pharmaceutically acceptable carrier.

47. A pharmaceutical composition comprising:
a compound according to claim 36 or a reduced sulfhydryl derivative of said compound; and
a pharmaceutically acceptable carrier.

48. A pharmaceutical composition comprising:
a compound according to claim 38; and
a pharmaceutically acceptable carrier.

49. A method of making a compound according to claim 29, said method comprising:
providing a benzopyran having the formula: 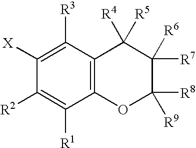
wherein X represents a hydroxy group or a protected hydroxy group; and
converting the benzopyran with a disulfide having the formula: 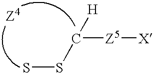
wherein X′ represents a carboxylic acid group or a protected carboxylic acid group under conditions effective to produce a compound according to claim 29.

50. A method according to claim 49, wherein each of R¹-R³ is a methyl group, wherein each of R⁴-R⁸ is hydrogen, and wherein R⁹ represents a substituted or unsubstituted lipophilic alkyl.

51. A method according to claim 50, wherein Z⁵ has the formula —(CH₂)ₙ— and wherein n is an integer from 3 to 5.

52. A method according to claim 51, wherein R⁹ is a 3,7,11-trimethyldodecyl group.

53. A method according to claim 52, wherein n is 4.

54. A method according to claim 53, wherein the compound has the formula:

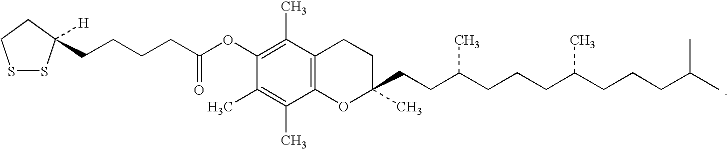

55. A method of inhibiting oxidative and/or free radical damage in a subject's cells, said method comprising administering a compound according to claim 1 or a reduced sulfhydryl derivative of said compound to the subject under conditions effective to inhibit oxidative and/or free radical damage in a subject's cells.

56. A method according to claim 55, wherein said administering is carried out enterally.

57. A method according to claim 55, wherein the oxidative and/or free radical damage is the result of lipid peroxidases.

58. A method according to claim 55, wherein the oxidative and/or free radical damage is the result of a calcium-activated protease.

59. A method according to claim 55, wherein the oxidative and/or free radical damage is the result of a calcium-activated lipase.

60. A method according to claim 55, wherein the oxidative and/or free radical damage is the result of lipid peroxidases.

61. A method according to claim 55, wherein the oxidative and/or free radical damage is the result of hypoxia.

62. A method according to claim 55, wherein the oxidative and/or free radical damage is the result of ischemia.

63. A method of inhibiting oxidative and/or free radical damage in a subject's nerve membranes, sarcoplasmic reticula, mitochondrial membranes, and/or muscle plasma membranes, said method comprising administering a compound according to claim 1 or a reduced sulfhydryl derivative of said compound to the subject under conditions effective to inhibit oxidative and/or free radical damage in the subject's nerve membranes, sarcoplasmic reticula, mitochondrial membranes, and/or muscle plasma membranes.

64. A method according to claim 63, wherein said administering is carried out enterally.

65. A method of treating and/or preventing obstructive bladder disease and/or ischemic bladder disease, said method comprising administering a compound according to claim 1 or a reduced sulfhydryl derivative of said compound to the subject under conditions effective to treat and/or prevent obstructive bladder disease and/or ischemic bladder disease.

66. A method according to claim 65, wherein said administering is carried out enterally.

67. A method of treating and/or preventing a condition involving ischemia, hypoxia, and/or reoxygenation injury in a subject, said method comprising administering a compound according to claim 1 or a reduced sulfhydryl derivative of said compound to the subject under conditions effective to treat and/or prevent the condition.

68. A method according to claim 67, wherein said administering is carried out enterally.

69. A method of inhibiting oxidative and/or free radical damage in a subject's cells, said method comprising administering a compound according to claim 38 or a reduced sulfhydryl derivative of said compound to the subject under conditions effective to inhibit oxidative and/or free radical damage in a subject's cells.

70. A method of inhibiting oxidative and/or free radical damage in a subject's nerve membranes, sarcoplasmic reticula, mitochondrial membranes, and/or muscle plasma membranes, said method comprising administering a compound according to claim 38 or a reduced sulfhydryl derivative of said compound to the subject under conditions effective to inhibit oxidative and/or free radical damage in the subject's nerve membranes, sarcoplasmic reticula, mitochondrial membranes, and/or muscle plasma membranes.

71. A method of treating and/or preventing obstructive bladder disease and/or ischemic bladder disease, said method comprising administering a compound according to claim 38 or a reduced sulfhydryl derivative of said compound to the subject under conditions effective to treat and/or prevent obstructive bladder disease and/or ischemic bladder disease.

72. A method of treating and/or preventing a condition involving ischemia, hypoxia, and/or reoxygenation injury in a subject, said method comprising administering a compound according to claim 38 or a reduced sulfhydryl derivative of said compound to the subject under conditions effective to treat and/or prevent the condition.
